(19) United States
(12) Patent Application Publication
(10) Pub. No.: US 2017/0196678 A1
(43) Pub. Date: Jul. 13, 2017

(54) TENDON AND LIGAMENT FIXATION DEVICE AND METHOD OF USE

(71) Applicant: University of Virginia Patent Foundation, Charlottesville, VA (US)

(72) Inventors: Joseph S. Park, Charlottesville, VA (US); David Chen, Charlottesville, VA (US); Jesse Seamon, Charlottesville, VA (US)

(21) Appl. No.: 15/467,480

(22) Filed: Mar. 23, 2017

Related U.S. Application Data
(63) Continuation-in-part of application No. PCT/US2015/051549, filed on Sep. 22, 2015.
(60) Provisional application No. 62/154,417, filed on Apr. 29, 2015.
(60) Provisional application No. 62/054,278, filed on Sep. 23, 2014.

(51) Int. Cl. A61F 2/08; A61B 17/17

(52) U.S. Cl. CPC A61F 2/0811; A61B 17/1714; A61B 2017/0409

(57) ABSTRACT

Devices and methods for tendon to bone healing are disclosed. In one embodiment, a tendon fixation method for tendon to bone healing includes anchoring suture anchors to a bone region. Sutures may be securely engaged to associated suture anchors and the sutures may be fastened to a fixation device. The fixation device may include a base with a plurality of openings to securely receive the sutures and one or more fastening members and/or fastening surfaces. The fixation device may be secured to a tendon thereby increasing a resistance to rotation or axial motion. In this respect, the contact area between the tendon and the bone region may be increased such that a compression force may be evenly distributed from the base of the fixation device along a tendon region of the tendon. In one embodiment, a plurality of elongate fastening members can be removably inserted through the opening until perforating a predetermined depth of the subchondral bone region and being stopped at the predetermined depth by contacting an upper surface of the base.

### CROSS-REFERENCE TO RELATED APPLICATIONS

This Application is a continuation-in-part of PCT International Patent Application Serial No. PCT/US2015/051549, filed Sep. 22, 2015, which claims priority to and benefit under 35 U.S.C §119(e) of U.S. Provisional Patent Application Ser. No. 62/054,278, filed Sep. 23, 2014 and U.S. Provisional Patent Application Ser. No. 62/154,417, filed Apr. 29, 2015, each of which are hereby incorporated by reference herein in their entirety as if fully set forth below.

This Application claims priority to and benefit under 35 U.S.C §119(e) of U.S. Provisional Patent Application Ser. No. 62/339,629, filed May 20, 2016, which is hereby incorporated by reference herein in its entirety as if fully set forth below.

### FIELD

The present disclosure generally relates to the treatment of tendon and ligament ruptures and avulsions, as well as tendon transfers intended to augment or replace dysfunctional tendons.

### BACKGROUND

Many clinical situations arise where a native tendon or ligament must be reinserted into its boney insertion site. Such situations may include biceps tendon avulsions, posterior tibial tendon reconstructions, accessory navicular, rotator cuff tears, Achilles tendon ruptures, or tearing of the anterior cruciate ligament (ACL). In general, two types of fixation predominate to treat tendon and ligament ruptures and avulsions: the use of bone tunnels and the use of suture anchors.

For bone tunnel techniques, a large diameter hole must be drilled through the bone at the tendon insertion site, and then a fixation option must be chosen to hold the tendon in the previously created tunnel. Frequently, the tunnels must be relatively large to accommodate the diameter of the tendon. This can be problematic in smaller bones such as the tarsal bones. Fixation may be achieved with bone tunnels with an interference screw that “presses” the tendon against the wall of the bone tunnel eccentrically on one side or with a “suspensory fixation” that typically involves a far cortical button device that suspends the tendon within the bone tunnel.

With respect to posterior tibial tendon (PTT) dysfunction, known treatment approaches include attaching a replacement tendon primarily to the navicular tuberosity to support the medial arch. In this approach, the PTT is replaced or augmented with the flexor digitorum longus (FDL) which has been known to exhibit approximately ¼ the strength of the PTT. Reference is made to FIGS. 25-26 depicting the problems experienced when routing the FDL through the navicular region to properly augment or replace the PTT during the required procedure. Specifically, FIG. 26 depicts an exemplary human foot from the top and from the side to clearly show positioning of the navicular bone with a typical interference screw. FIG. 20 depicts a side plan view of a human foot with the PTT having been cut and positioned adjacent the FDL. The FDL can be seen having formed a bone tunnel within the navicular bone. Similarly, FIG. 27 clearly shows the interplay between PTT and FDL as described and difficulties experienced by those skilled in the art when accessing the PTT to augment or replace it with the FDL.

In practice, the FDL may be attached by being tied back to itself but this may be problematic for several reasons. First, a longer incision is required and morbidity is increased from the FDL harvest as clearly seen in FIG. 25. Second, there is an increased concern for bone tunnel blowout or navicular fracture since use of large diameter drills are required to create the required tunnel to then augment or replace the PTT.

Another approach includes the use of interference screws. These may be preferable to PTT augmentation as described since they require a shorter tendon harvest and yet, interference screws have several conspicuous drawbacks. First, uneven or asymmetric compression results. Second, a larger hole is required for their implantation as depicted, for example, in FIG. 26. Additionally, the resultant bone tunnel has been known to expand as a consequence of motion, which can lead to osteolysis. Finally, the risk of fracture increases due to the increased forces required to insert the screw.

Bone tunnels as described are therefore associated with several noted complications including tunnel osteolysis, tunnel blowout, and fractures at the tunnel site. In addition, since the tendon to be repaired has to be pulled into a tunnel, over-tensioning is a significant concern in addition to prolonged swelling and increased pain experienced by patients. The bone tunnel does, however, supply a large surface area of bleeding bone to promote tendon integration and provides a relatively long conduit for fixation.

For suture anchor techniques, relatively smaller holes are drilled at that tendon insertion site and then anchors that have an integrated suture through an eyelet are inserted into bone. As a practical matter, because the holes are relatively smaller with suture anchors, the swelling and bone pain is relatively lower as well. The suture is passed through the tendon and ties the tendon down directly to bone at that point. Multiple anchors can be used to replicate the insertional anatomy, but ultimately, tendon to bone apposition only occurs at the suture anchor sites. Such suture anchoring can also be enhanced with suture bridge techniques (“or double row”) but it is difficult, if not impossible, to provide evenly directed compression across the entire area of the tendon on its anatomic foot print. This is especially problematic for smaller bones such as the navicular tuberosity.

With suture anchors, each fixation point is small and is reliant on the suture grasping the tendon. Therefore, in poorer quality tissue, the suture anchor technique alone can be particularly problematic with the suture fixation point tearing a longitudinal rent in the tendon before failure, often dubbed the “cheese cutter” effect as described in Burkhart et al., Biomechanical validation of load-sharing rip-stop fixation of tissue deficient rotator cuff tears, Am J Sports Med. 2014; 42(2):457-62. In poor quality bone or less dense areas of normal bone, suture anchor pull-out can be a problem as described in Barber et al., The relationship of suture anchor failure and bone density to proximal humerus location: a cadaveric study. Arthroscopy. 1997: 13(3);340-45. The suture anchor technique does allow one to accurately replicate the insertional footprint of a tendon, especially when multiple anchors are used. Nevertheless, actual bone-tendon contact will only be at suture anchors sites.

Studies have shown that in general, suspensory and interference screw fixation in bone tunnels may be slightly stronger than suture anchor fixation in regards to pull out strength. However, the difference in pull out strength is usually small and results have been contradictory. Accordingly, the clinical implications of the observed differences are not clear.

One common disadvantage to both techniques is the inability to reliably promote osseous integration of tendon into bone. Suture anchor techniques allow a small surface area for tendon to bone healing whereas suspensory fixation in bone tunnels can allow for significant tendon motion within the tunnel and subsequent osteolysis and lack of adequate tendon integration. Interference screw fixation eccentrically forces the tendon into one side of the bone tunnel decreasing the effective surface area for boney integration and warping the tendon at the site of bone integration. Common to both bone tunnel fixation techniques is the inability to truly restore the anatomic footprint of the inserted tendon or reconstructed ligament.

Several in vitro animal studies have looked at the use of biocompatible scaffolds at the tendon insertion site with favorable histologic and biomechanical results when compared to standard techniques. See, e.g., Yokoya et al., Tendon-bone insertion repair and regeneration using polyglycolic acid sheet in the rabbit rotator cuff injury model. Am J Sports Med. 2008:35(7):1298-1309; and Kadonishi et al., Acceleration of tendon-bone healing in anterior cruciate ligament reconstruction using an enamel matrix derivative (EMD) in a rat model. J Bone Joint Surg Br. 2012;94(2):205-9; and Moffat et al., Orthopedic interface tissue engineering for the biological fixation of soft tissue grafts. Clin Sports Med. 2009:28(1):157-76.

It is with respect to these and other considerations that the various embodiments described below are presented.

### SUMMARY

In some aspects, the present disclosure relates to tendon and ligament fixation devices and related methods of use. In some embodiments, one or more devices may be constructed from at least one metal or a combination of metal with porous metal scaffold that may perforate the subchondral bone and may exhibit structure similar to cancellous bone. One or more devices in accordance with some embodiments of the present disclosure may also be constructed from other osteoconductive metal constructs such as plasma spray metal, grit blasted cobalt chrome, and/or hydroxyapatite (HA) coated metal depending on respective efficacy at tendon insertion sites. In some embodiments, the fixation device may also be made of inert plastic, bio-compatible material, or other polymer with design features that minimize the prominence of the implant and irritation of the overlying skin. Such a modular device would accommodate concurrent use of modular porous metal spikes or screws/nails that perforate bone and allow for boney fixation. As demonstrated in FIG. 30, these modular elements would provide rotational control, improved compression, and biologic fixation to the underlying bone.

One or more devices according to some embodiments of the present disclosure may provide biomechanically sound fixation to allow for early protected motion, accurate restoration of the anatomic footprint of the tendon or reconstructed ligament, and may promote osseous integration of the tendon at the insertion site. In some embodiments, a tendon fixation method for tendon to bone healing is disclosed. The method can include anchoring suture anchors to a bone region. Sutures may be securely engaged to associated suture anchors and the sutures may be fastened to a fixation device. In some embodiments, a fixation device may include a base with a plurality of openings to securely receive the sutures and one or more fastening members and/or fastening surfaces. The fixation device may be secured to a tendon thereby increasing a resistance to rotation or axial motion. In this respect, the contact area between the tendon and the bone region may be increased such that a compression force may be evenly distributed from the base of the fixation device along a tendon region of the tendon.

The suture anchors may be anchored by utilizing an implantation device such as a drill or a planer. In some embodiments, the above described methods may promote osseous integration of the tendon at an insertion site, and the sutures may be tied over the fixation device. The bone region may also be prepared to a predetermined depth prior to anchoring suture anchors. The predetermined depth may correspond to a thickness of the fixation device. Securing the fixation device to the tendon may include stabilizing the fixation device by penetrating the tendon with the one or more fastening members and/or perforating the bone region with the one or more fastening members. In some embodiments, the method may also include selectively positioning the openings of the base to maximize the compression force.

In some embodiments, the fixation device may include a curve or contoured surface in communication with the base operable to conform to a contour of the bone region. In this respect, the curve of the fixation device may be constructed with the pre-defined contour or may be selectively adjusted between a plurality of orientations ranging between substantially planar to substantially curved. The bone region may preferably be the navicular and the tendon is the flexor digitorum longus.

In some embodiments, a tendon fixation method may include cleaving the tendon with one or more of the fastening members, the one or more fastening members being a cleat, a spike, a barb or the like such that the member may be operable to perforate the bone region. Alternatively, the fastening member(s) may be a protrusion, tine, cone, stud, pedestal, pin, projection, or knurling. The tendon may be cleaved with at least two of the fastening members and traction may be induced with the tendon during a reattachment process with a plurality of remaining fastening members relatively smaller than the fastening members used for cleaving. In this respect, the remaining fastening members may be selectively positioned in a predetermined array in one or more regions of the base portion between or adjacent the fastening members capable of cleaving the tendon.

In some embodiments, the fixation device itself may exhibit material properties similar to cancellous bone and may be constructed from one or a combination of a porous metal, titanium, porous titanium, tantalum, biofoam, polyether ether ketone, plastic, thermoplastic, polymer, metal, trabecular metal, alloy, stainless steel, polylactic acid or polylactide, or silicon. In addition, in some embodiments, the implant may be bio-printed using materials allowing for incorporation of the fixation device, including collagen or other bio-compatible substance.

The one or more fastening surfaces may include a plurality of ridges, grooves, or protrusions capable of inducing traction with the tendon. The ridges, grooves, or protrusions may preferably be disposed on a lower surface of the base and may be oriented parallel and/or orthogonal with lateral edges of the base portion.

In some embodiments, the fixation device may also be partially or completely coated with a medium. The medium may include growth factors, a pharmaceutically active substance, cells, fluids, biological fluids, drugs, gene therapy vectors, irrigation fluids, growth factors, nuclear medicine agents, antibiotics, anti-viral agents, contrast agents, chemotherapies, or other diagnostic or therapeutic agents. Coating of the medium on the fixation device may include selectively coating one or more predetermine regions of the fixation device.

In some embodiments, the fixation device may be imageable and/or imaged using a medical imaging modality. The modality may include radiography, fluoroscopy, magnetic resonance imaging, medical ultrasonography or ultrasound, endoscopy, elastography, tactile imaging, thermography, tomography, echocardiography, medical photography or the like.

The resistance to rotation may be increased and the toggle of the fixation device reduced by securing the fixation device on top of the tendon region. Bony ingrowth may be promoted through or around the tendon and the tendon may be reattached to the bone region.

In some embodiments, a fixation device for bone to tendon healing may include a base with a plurality of openings for detachably fastening with a plurality of sutures as well as a plurality of fastening members operable to securely engage with the tendon to increase a resistance of rotation or axial motion. In this respect, the base may be capable of evenly distributing a compression along a tendon region of the tendon. The tendon may be detachably fastenable to a pair of suture anchors with one or more of the fastening members extending outwardly from the base and being capable of perforating a bone region.

As the base portion may include a contour or curvature, this contour or curvature may be rigidly constructed in the base portion or the contour or curvature may be adjustable between a plurality of orientations ranging between substantially planar to substantially curved.

In some embodiments, the fastening members may be capable of cleaving the tendon and extend outwardly from the base portion, wherein each member may have a proximal portion adjacent the base and a distal portion capable of cleaving the tendon. The distal portion may be tapered from the proximal portion to a pointed distal end capable of cleaving the tendon and/or perforating the bone region. One or more of the fastening members may be a cleat, spike, barb, protrusion, tine, cone, stud, pedestals, pins, projection, or knurling. In some embodiments, at least two of the fastening members may be capable of cleaving the tendon whereas the remaining fastening members may be relatively smaller and capable of inducing traction with the tendon. The remaining fastening members may be positioned in a predetermined array in one or more regions of the base portion between or adjacent the fastening members capable of cleaving the tendon.

One or more of the fastening members may be integrally formed with the base or detachably connected thereto. A fixation device according to some embodiments may also include a fastening surface operable to induce traction with the tendon. The fastening surface may include a plurality of ridges, grooves, or protrusions capable of inducing traction with the tendon. The ridges, grooves, or protrusions may preferably be disposed on a lower surface of the base and may be oriented parallel and/or orthogonal with lateral edges of the base portion.

In some embodiments of the present disclosure, a fixation device may include a base with a plurality of openings for detachably fastening with a plurality of sutures and only a fastening surface in communication with the base for inducing traction with the tendon to increase a resistance of rotation or axial motion. The fastening surface may be defined by a frictional cleated surface defined by one or more friction inducing ridges or grooves operable to induce traction with the tendon. The ridges or grooves may also be constructed from differing depths and each ridge or groove may orthogonal, diagonal or in parallel with respective lateral side edges of the base. Alternatively, the ridges or grooves of member may be defined by a plurality of concentric circles, ellipses, or rectangles. Fastening members and coating as previously described may also be included with this fixation device and corresponding fastening surface.

In some embodiments of the present disclosure, a tendon fixation method is provided, which comprises: anchoring at least one suture anchor to a bone region; securely engaging sutures to the at least one suture anchor; attaching a fixation device to the bone region; fastening the sutures to eyelets of the fixation device, the fixation device being capable of movement in a controlled range of motion; and removably inserting a plurality of fastening members through the eyelets of a base of the fixation device to a predetermined depth with respect to the bone region thereby securing the fixation device to a tendon to thereby increase a resistance to rotation or axial motion. In some embodiments, the at least one suture anchor is anchored using an implantation device, the implantation device being a drill or a planer, the method optionally comprising: increasing contact area between the tendon and the bone region by distributing a compression force evenly from the base of the fixation device along a tendon region of the tendon. In some embodiments, the method provides for promoting osseous integration of the tendon at an insertion site and/or preparing the bone region to the predetermined depth prior to anchoring the at least one suture anchor.

In some embodiments, each fastening member comprises an elongated member having distal and proximal portions, the distal portion comprising a piercing portion operable to pierce a tendon and the proximal portion comprising a washer surface wider than a remainder of the fastening member, the washer of the proximal portion operable to stop the fastening member once inserted to the predetermined depth. In some embodiments, the method comprises selectively removing a portion of the washer, thereby forming an access for the sutures and/or forming one or more knots with the sutures on top of the fixation device. In some embodiments, the washer is removably attached to the fastening member. In some embodiments, the method further comprises forming a plurality of gripping fastening members on a lower surface of the base of the fixation device. In some embodiments, each fastening member is constructed from porous metal with a grip inducing external coating optimized for osseous integration through and around the tendon. In some embodiments, the fixation device is secured to the tendon by the fastening members being inserted through the openings of the base and perforating and/or capturing a subchondral bone to promote osseous integration.

In some embodiments, the method comprises selectively adjusting a curve of the fixation device between a plurality of orientations ranging between substantially planar to substantially curved. In some embodiments, the bone region is the navicular and the tendon is the flexor digitorum longus, and wherein the base of the fixation device is conformable to the navicular bone. In some embodiments, the fixation device is constructed from one or a combination of a porous metal, titanium, porous titanium, tantalum, biofoam, polyether ether ketone, plastic, thermoplastic, polymer, metal, trabecular metal, alloy, stainless steel, polylactic acid or polylactide, or silicon.

In some embodiments, the method comprises cleaving the tendon with one or more of the fastening members, wherein the one or more fastening members comprise at least one of a cleat, a spike, or barb operable to perforate the bone region. In some embodiments, the method comprises cleaving the tendon with one or more of the fastening members, wherein the one or more fastening members comprises at least one of a protrusion, tine, barb, cone, stud, pedestals, pins, projection, or knurling. In some embodiments, the method comprises selectively positioning the eyelets of the base to maximize the compression force. The method of any of the preceding claims, further comprising: positioning a fastening surface on a lower surface of the base, the fastening surface comprising a plurality of ridges, grooves, or protrusions capable of inducing traction with the tendon. In some embodiments, the method comprises coating the fixation device with a medium, the medium optionally comprising at least one of growth factors, a pharmaceutically active substance, cells, fluids, biological fluids, drugs, gene therapy vectors, irrigation fluids, growth factors, nuclear medicine agents, antibiotics, anti-viral agents, contrast agents, chemotherapies, or other diagnostic or therapeutic agents. In some embodiments, coating the fixation device includes selectively coating one or more predetermined regions of the fixation device.

In some embodiments, the method comprises imaging the fixation device using a medical imaging modality, the medical imaging modality optionally including at least one of radiography, fluoroscopy, magnetic resonance imaging, medical ultrasonography or ultrasound, endoscopy, elastography, tactile imaging, thermography, tomography, echocardiography, or medical photography. In some embodiments, the fixation device is constructed from one or a combination of titanium, porous titanium, or biofoam. In some embodiments, the fixation device has cancellous bone material properties.

In some embodiments, the method comprises cleaving the tendon with at least two of the fastening members when removably inserting each fastening member; and inducing traction with the tendon during a reattachment process with a plurality of remaining fastening members relatively smaller than the fastening members used for cleaving.

In some embodiments of the present disclosure, a tendon fixation device is provided, which comprises a base with a plurality of openings for detachably fastening with a plurality of sutures and receiving a plurality of elongate fastening members; and the plurality of elongate fastening members operable to be removably inserted through a respective opening of the base and perforate a subchondral bone region when the base is coupled thereto until a predetermined depth on top of the base, wherein the base is capable of evenly distributing a compression force along a tendon region of the tendon. In some embodiments, the elongate fastening members are capable of piercing the tendon, each elongate fastening member being formed from porous metal and being substantially elongate and divided by distal and proximal portions, the distal portion comprising a piercing portion operable to pierce a tendon and the proximal portion comprising a washer surface wider than a remainder of the fastening member, the washer of the proximal portion operable to be attached onto the base and stop the fastening member when inserted to the predetermined depth, the predetermined depth being defined by the subchondral bone region and/or through and around the tendon.

In some embodiments, the washer includes a selectively removed portion thereby forming an access for the sutures and/or an access for forming one or more knots with the sutures on top of the fixation device. In some embodiments, the washer is removably attached to the fastening member. In some embodiments, the distal portion of one or more of the fastening members comprises at least one of a cleat, a spike, or a barb. In some embodiments, the distal portion of one or more of the fastening members comprises at least one of a protrusion, tine, cone, stud, pedestals, pins, projection, or knurling. In some embodiments, the base portion comprises a curvature, the curvature being optionally adjustable between a plurality of orientations ranging between substantially planar to substantially curved.

In some embodiments of the present disclosure, the device comprises a plurality of gripping fastening members on a lower surface of the base of the fixation device. In some embodiments, the device comprises a coating externally positioned on the base and/or the fastening members, the coating optionally comprising at least one of growth factors, a pharmaceutically active substance, cells, fluids, biological fluids, drugs, gene therapy vectors, irrigation fluids, growth factors, nuclear medicine agents, antibiotics, anti-viral agents, contrast agents, chemotherapies, or other diagnostic or therapeutic agents.

In some embodiments, the bone region is the navicular and the tendon is the flexor digitorum longus, and wherein the base is conformable to the navicular bone In some embodiments, the fixation device is constructed from one or a combination of a porous metal, titanium, porous titanium, tantalum, biofoam, polyether ether ketone, plastic, thermoplastic, polymer, metal, trabecular metal, alloy, stainless steel, polylactic acid or polylactide, or silicon. In some embodiments, each opening is evenly spaced in opposing halves of the base. In some embodiments, the device comprises a fastening surface positioned on a lower surface of the base, the fastening surface comprising a plurality of ridges, grooves, or protrusions capable of inducing traction with the tendon. In some embodiments, the ridges, grooves, or protrusions are disposed on a lower surface of the base and are oriented parallel and/or orthogonal with lateral edges of the base portion. In some embodiments, each elongate member comprises a proximal portion in communication with the base and a distal portion tapering to a point capable of cleaving the tendon and/or perforating the bone region.

Other aspects and features of the present disclosure will become apparent to those of ordinary skill in the art, upon reviewing the following detailed description in conjunction with the accompanying figures.

### BRIEF DESCRIPTION OF THE DRAWINGS

Reference will now be made to the accompanying drawings, which are not necessarily drawn to scale.

FIG. 1 depicts a side plan view of a fixation device in accordance with one embodiment of the present disclosure.

FIG. 2 depicts an upper perspective of the device of FIG. 1 showing eyelets on the base portion.

FIG. 3 depicts a forward perspective of the device of FIG. 1 similarly showing eyelets on the base portion.

FIG. 4 depicts a top plan view of the device of FIG. 1 showing the eyelets with respect to the base portion.

FIG. 5 depicts a side plan view of a fixation device in accordance with one embodiment of the present disclosure wherein the base portion is planar.

FIG. 6 depicts an upper perspective of the device of FIG. 5 showing eyelets on the base portion of the fixation device.

FIG. 7 depicts an upper perspective view of a fixation device in accordance with one embodiment of the present disclosure with a plurality of fastening members.

FIG. 8 depicts a bottom plan view of the device of FIG. 7 showing the location of exemplary fastening members.

FIG. 9 depicts a side plan view of the device of FIG. 7 showing the contour of the device and associated fastening members.

FIG. 10 depicts a lower perspective view of a fixation device in accordance with one embodiment of the present disclosure with a lower fastening member surface.

FIG. 11 depicts a side plan view of the device of FIG. 10.

FIG. 12 depicts a lower perspective view of a fixation device in accordance with one embodiment of the present disclosure having a similar fastening member surface as that shown in FIG. 10.

FIG. 13 depicts a bottom plan view of the device of FIG. 12.

FIG. 14 depicts a fixation device in accordance with one embodiment of the present disclosure

FIG. 15 depicts a side plan view of the fixation device of FIG. 14.

FIG. 16 depicts a lower perspective view of a fixation device in accordance with one embodiment of the present disclosure with both fastening members and fastening member surface.

FIG. 17 depicts a side plan view of the device of FIG. 16.

FIG. 18 depicts an upper perspective view of a fixation device in accordance with one embodiment of the present disclosure, depicting a pair of eyelets and combination of different sized fastening members.

FIG. 19 depicts a bottom plan view of the device of FIG. 18 showing the location of the different sized exemplary fastening members.

FIG. 20 depicts a schematic overview of a method of using a fixation device, in accordance with an embodiment of the present disclosure.

FIG. 21 depicts a schematic overview of an exemplary implementation of fixation devices in accordance with some embodiments of the present disclosure.

FIG. 22 depicts a schematic overview of another exemplary implementation for load and displacement evaluation of fixation devices in accordance with some embodiments of the present disclosure.

FIG. 23 is a graphical depiction of load versus extension for the implementation of FIG. 22.

FIGS. 24A and 24B are a binary depiction of images showing pressure area difference between the control and device conditions of FIGS. 22-23.

FIG. 25 depicts a side view of an exemplary human foot with a PTT that has been cut and FDL tendon that has been routed through the navicular region.

FIG. 26 depicts side and top views of an exemplary human foot depicting an exemplary interference screw positioned with the navicular region.

FIG. 27 depicts an exemplary view of a surgeon accessing the PTT and FDL.

FIGS. 28A and 28B depict x-ray images of side plan views of exemplary human feet, wherein FIG. 28A depicts a foot without interference screws or suture anchors whereas FIG. 28B depicts a foot with interference screws and suture anchors.

FIGS. 29A and 29B depict x-ray images of top plan views of exemplary human feet similar to FIGS. 28A and 28B, wherein FIG. 29A depicts a foot without interference screws or suture anchors, whereas FIG. 29B depicts a foot with interference screws and wedges, wherein FIG. 29A depicts a foot having a severe flatfoot deformity without interference screws or suture anchors, and whereas FIG. 29B depicts a foot with osteotomies, wedges, a bone tunnel, and interference screws.

FIG. 30 depicts an upper perspective view of a fixation device in accordance with one embodiment of the present disclosure.

FIG. 31 depicts a lower plan view of the fixation device of FIG. 30.

FIG. 32 depicts a lower perspective of the device of FIG. 30.

FIG. 33A depicts a schematic overview of a first step of a method of using a fixation device, in accordance with an embodiment of the present disclosure.

FIG. 33B depicts a schematic overview of a second step of the method of FIG. 33A, in accordance with an embodiment of the present disclosure.

FIG. 34 depicts a schematic overview of an exemplary implementation of fixation devices in accordance with some embodiments of the present disclosure.

FIG. 35 depicts a schematic overview of another exemplary implementation for load and displacement evaluation of fixation devices in accordance with some embodiments of the present disclosure.

FIG. 36 is a photograph of an implanted device in accordance with the some embodiments of the present disclosure, involving a flexor digitorum longus transfer to the rabbit tibia.

### DETAILED DESCRIPTION

Although example embodiments of the disclosed technology are explained in detail herein, it is to be understood that other embodiments are contemplated. Accordingly, it is not intended that the disclosed technology be limited in its scope to the details of construction and arrangement of components set forth in the following description or illustrated in the drawings. The disclosed technology is capable of other embodiments and of being practiced or carried out in various ways.

It must also be noted that, as used in the specification and the appended claims, the singular forms “a,” “an” and “the” include plural referents unless the context clearly dictates otherwise. By “comprising” or “containing” or “including” it is meant that at least the named compound, element, particle, or method step is present in the composition or article or method, but does not exclude the presence of other compounds, materials, particles, method steps, even if the other such compounds, material, particles, method steps have the same function as what is named.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the disclosed technology. Similarly, it is also to be understood that the mention of one or more components in a device or system does not preclude the presence of additional components or intervening components between those components expressly identified.

In the following description, references are made to the accompanying drawings that form a part hereof and that show, by way of illustration, specific embodiments or examples. In referring to the drawings, like numerals represent like elements throughout the several figures.

As discussed herein, a “subject” or “patient” may be a human or any animal. It should be appreciated that an animal may be a variety of any applicable type, including, but not limited thereto, mammal, veterinarian animal, livestock animal or pet type animal, etc. As an example, the animal may be a laboratory animal specifically selected to have certain characteristics similar to a human (e.g., rat, dog, pig, monkey, or the like). It should be appreciated that the subject may be any applicable human patient, for example.

In certain embodiments of the present disclosure, a fixation device capable of being tied over a suture anchor may be provided to facilitate reliable tendon to bone healing as well as significantly improve in compression forces at the tendon-bone junction. In some embodiments, the fixation device is capable of increasing the contact area at the tendon-bone block interface and increases strength of the interface connection. Fixation devices according to various embodiments of the present disclosure described herein may have one, a pair, or any number of fastening members and/or fastening member surfaces that, in combination with the corresponding base portion, will allow for elimination of “toggle” and promote biologic healing of bone through and around the tendon. Since full biologic healing of tendon to bone may take more than 26 weeks, early and maintained rigid fixation at this interface is critical to ensure maximal strength of the tendon insertion. Preferably, tendons repaired using the herein disclosed fixation device will heal closer to their original structure with less scar tissue formation than tendons repaired using known suture anchor techniques.

In certain embodiments of the present disclosure, a fixation device is disclosed that is capable of achieving reliable tendon to bone healing for successful treatment of various tendon disorders. A fixation device according to embodiments disclosed herein may be capable of movement in a controlled range of motion to improve collagen organization and tensile strength. In some embodiments, the fixation device may be capable of delivering greater compressive force between the tendon and insertion site to prevent severing of tendon and promote osseous integration. In this regard, a fixation device may capable of reducing the risk of the tendon being sliced by one or more suture anchors under physiologic load. In some embodiments, a fixation device disclosed herein can be capable of being tied over a suture anchor to facilitate reliable tendon to bone healing as well as significantly improve in compression forces at the tendon-bone junction. In some embodiments, the fixation device is capable of increasing the contact area at the tendon-bone block interface and increases strength of the interface connection.

A fixation device according to some embodiments of the present disclosure may be constructed from at least one metal and/or metal coating. One particularly suitable, non-toxic metal is elemental tantalum scaffold that exhibits substantially similar multi-dimensional structure to cancellous bone such as Trabecular Metal® (manufactured by Zimmer, Inc., Warsaw, Ind., USA). Other osteoconductive metal constructs are also contemplated to be used including porous titanium, plasma sprayed metal, grit blasted cobalt chrome, HA coated metal, or the like depending upon respective efficacy at tendon insertion sites. Trabecular metal may be effective at promoting osseous ingrowth in the total joint replacement literature and positive results in terms of boney ingrowth may be obtained with other osteoconductive metals constructs discussed herein or otherwise known in the total joint literature. Iterations of the device which do not penetrate cortical bone may be made of inert polymers, plastics, or metals, depending on the application. A device according to some embodiments of the present disclosure can also be made of another degradable or inert polymer that may elute substances, including growth factors, which can stimulate boney ingrowth. Iterations of the device which do not penetrate cortical bone may be made of inert polymers, plastics, or metals, depending on the application. These iterations can accommodate modular pegs made of a porous metal or similar material which may be passed through the eyelets, through the tendon, and into the underlying bone.

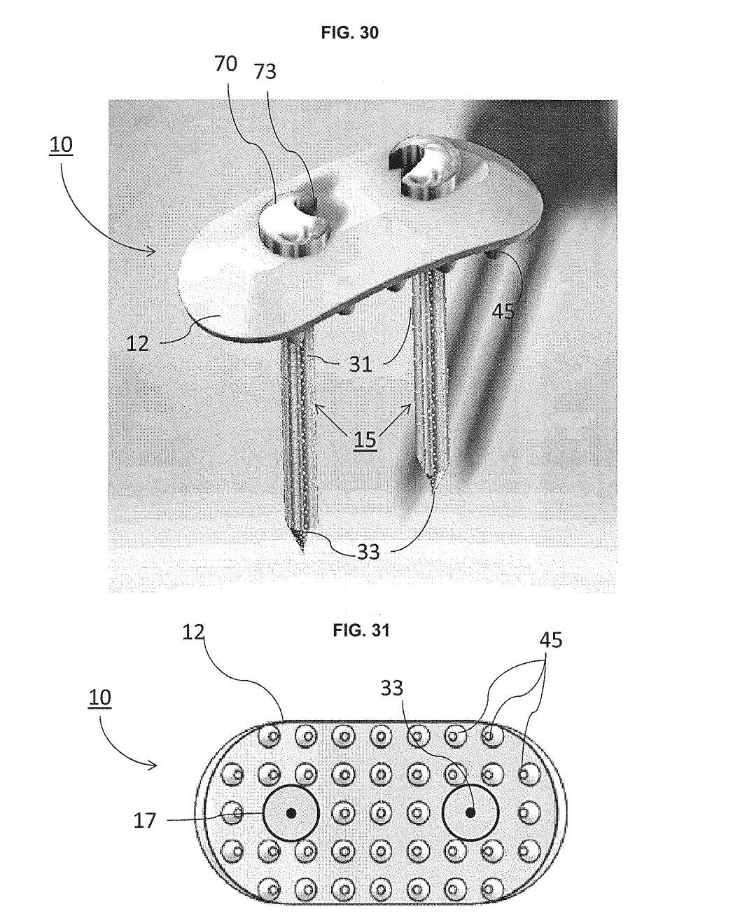

In some embodiments, the fixation device may also be made of inert plastic, bio-compatible material, or other polymer with design features that minimize the prominence of the implant and irritation of the overlying skin. Such a modular device would accommodate concurrent use of modular porous metal spikes or screws/nails that perforate bone and allow for boney fixation. As demonstrated in FIG. 30, these modular elements would provide rotational control, improved compression, and biologic fixation to the underlying bone. In addition, the implant may be bio-printed using materials allowing for incorporation of the fixation device, including collagen or other bio-compatible substance.

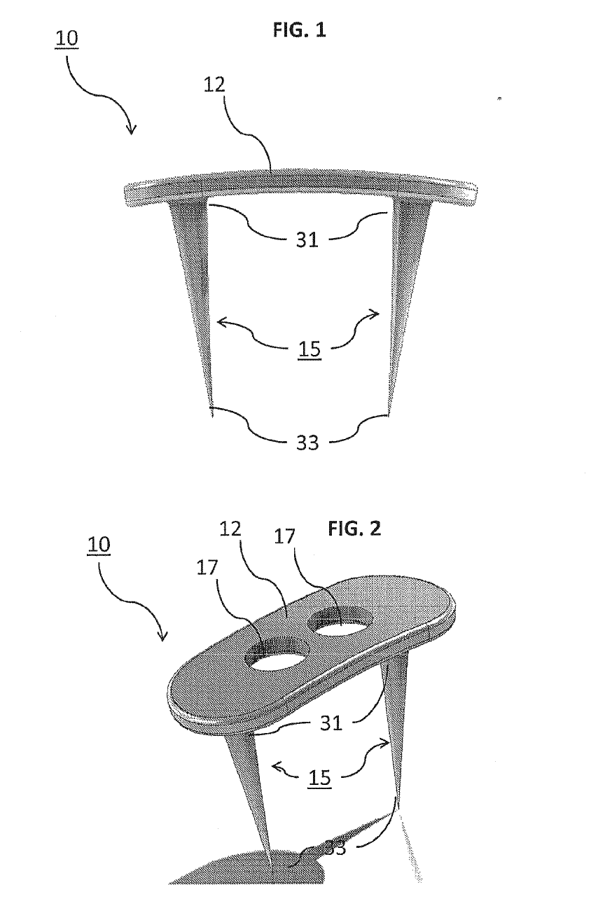

Turning to FIG. 1, a side plan view of fixation device 10 according to one embodiment of the present disclosure is depicted. In this embodiment, device 10 may be constructed from porous metal and be operable to securely engage with a tendon 20 (see e.g. FIG. 22 et seq.) through a friction and/or cleat engagement as between base portion 12 of device 10 and its pair of outwardly extending fastening members 15. This novel engagement may be operable to stimulate bone ingrowth through and around tendon 20 while also promoting biologic healing for permanent tendon-bone fixation. Furthermore, device 10 is capable of reducing the “toggle” and rotation of device 10 once secured on top of tendon 20.

Members 15 may be operable to pierce tendon 20 and/or go outside the tendon 20. Device 10, base 12, members 15, and/or eyelets 17 (see e.g. FIG. 2 et seq.) may comprise any number of sizes, dimensions, contours, rigidity, shapes, flexibility and materials of any of the components or portions of components in the various embodiments of the device discussed throughout. Likewise each of the foregoing features of device 10 may be varied and utilized as desired or required including size, material properties, predetermined locations of device 10 and/or alignments of the various components of or with device 10 may vary as desired or required.

Each of the members 15 in this embodiment commences with a wider, thicker proximal portion 31 and tapers to a distal pointed portion 33 so as to be able to secure device 10 to tendon 20 by, for example, a friction or cleat engagement by piercing the corresponding tendon 20 with each of members 15. Base 12 may be flexible or resilient so that base 12 may be adjusted or capable of conforming to the corresponding tendon 20 or ligament during implantation. Device 10 may also be relatively rigid and/or curved or contoured in a manner consistent with the corresponding tendon 20. Members 15 as depicted may be outwardly extending spikes or barbs that are capable of piercing corresponding tissue and securely engaging device 10 in place during implantation. In some embodiments, members may be capable of perforating the bone to further stabilize device 10 during use. Preferably, device 10 may comprise porous metal or other materials to permit bony ingrowth.

Each member 15 of FIG. 1 may measure 5 mm in length and/or diameter but device 10 is not so limited and said dimensions of member 15 may be lesser or greater than 5 mm as needed or required by the desired application. Further, base portion 12 of device 10 may measure 8 mm in length and 3.5 mm in width though these dimensions may vary with either or both dimensions being relatively or substantially larger or smaller as needed or required. Shapes and sizes will depend on the intended bone targeted for tendon reattachment. Similarly, number of eyelets can be increased for irregular or larger tendon footprints to increase fixation strength.

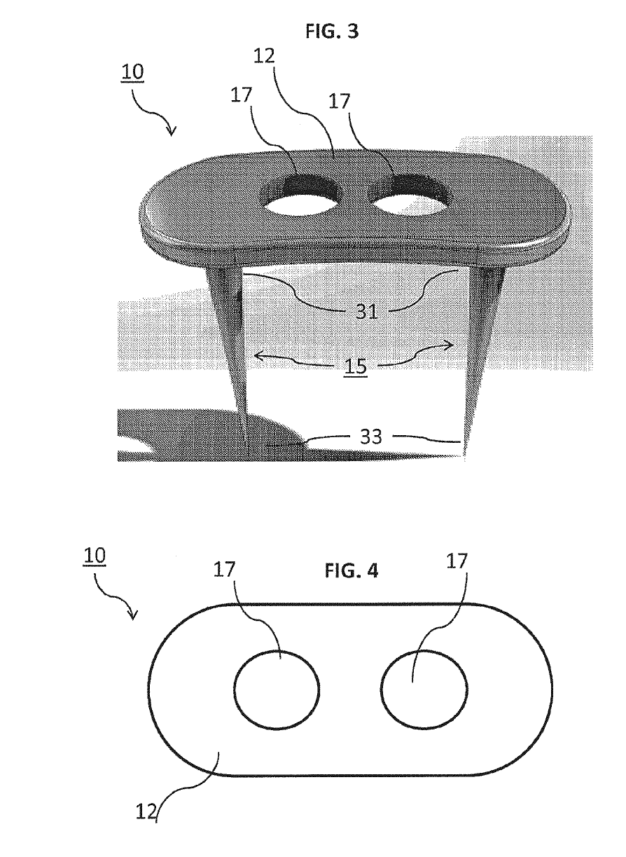

FIGS. 2-3 depict upper perspective views of device 10 of FIG. 1, wherein a pair of eyelets 17 are depicted on base 12 providing a portal or window between the upper and lower surfaces of base 12 to receive suture 13 (see e.g. FIG. 20 et seq.) or the like. While only two eyelets 17 are depicted in FIGS. 1-3, device 10 is not so limited and any number of eyelets 17 may be provided in any manner of shapes and sizes including circular, elliptical, rectangular or the like. Furthermore, spacing or positioning of eyelets 17 may be optimized to maximize compressive force delivered to tendon 20. FIG. 4 depicts a top plan view of base 12 of device 10 with the illustrated positioning of eyelets 17 thereon. Similarly, FIGS. 1-3 depict only two members 15 positioned opposite each other on opposing portions of base 12 and yet device 10 is not limited. Device 10 may include only one member 15 or any number of members 15 extending from base 12. Members 15 may also be positioned in one or more arrays or patterns on the lower surface 12 to for securely engage device 10 with corresponding tendon 20. As can be seen, device 10 when used with tendon 20 results in greater compressive force between tendon 20 and its insertion site thereby preventing the severing of tendon 20. Shape of base 12 may range between circular, elliptical, rectangular, or any shape as desired or needed or some combination of shapes as is the case in exemplary base 12 of FIGS. 1-4.

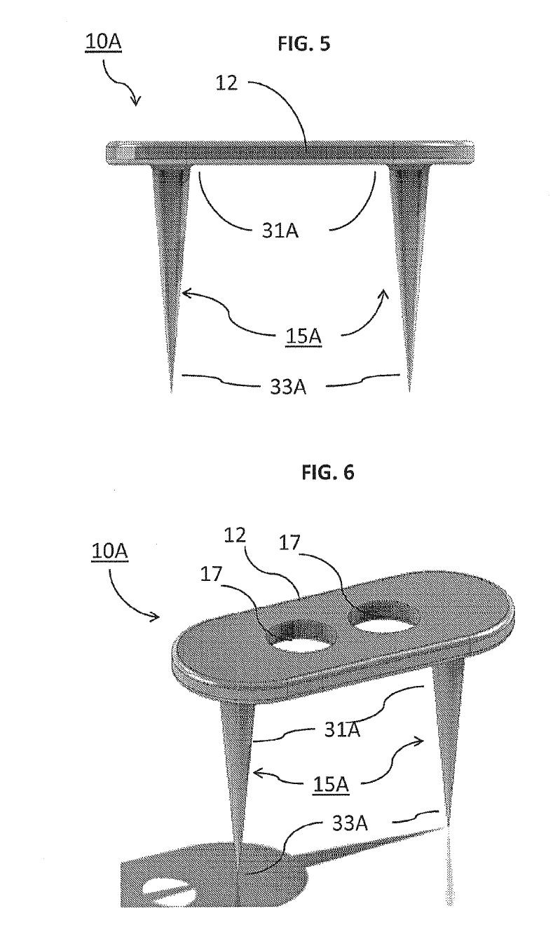

Turning to FIG. 5, device 10A can now be seen with base 12 being substantially planar or flattened. In this respect, base 12 may be relatively flexible and capable of flexing as in FIGS. 1-4. Base 12 of FIG. 5 may also be relatively rigid and thus resistant to bending or being forced out of its substantially planar shape. The ultimate shape and rigidity of the construct will again depend on the shape and location of the bone of interest. FIG. 6 depicts an upper perspective view of device 10 more clearly showing the planar contour of base 12 of device 10.

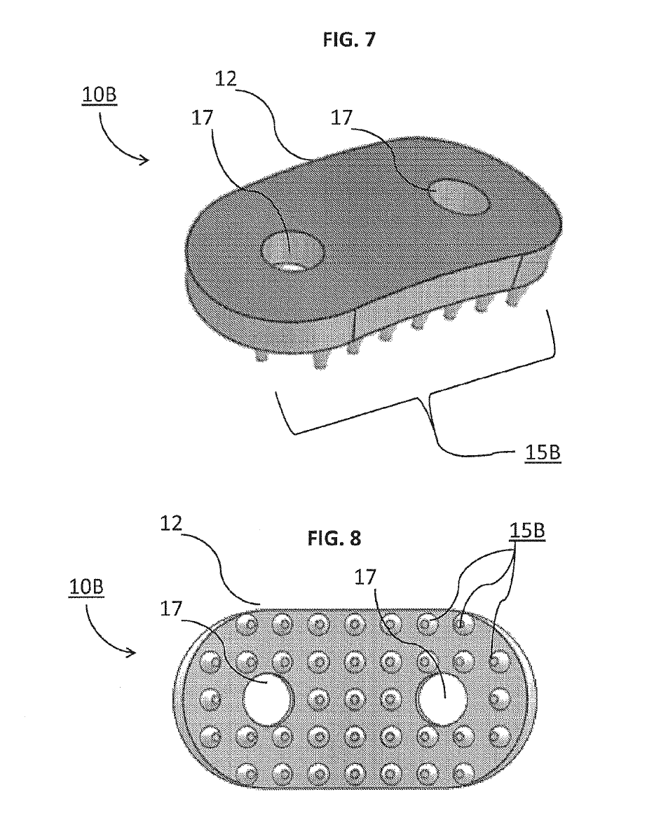

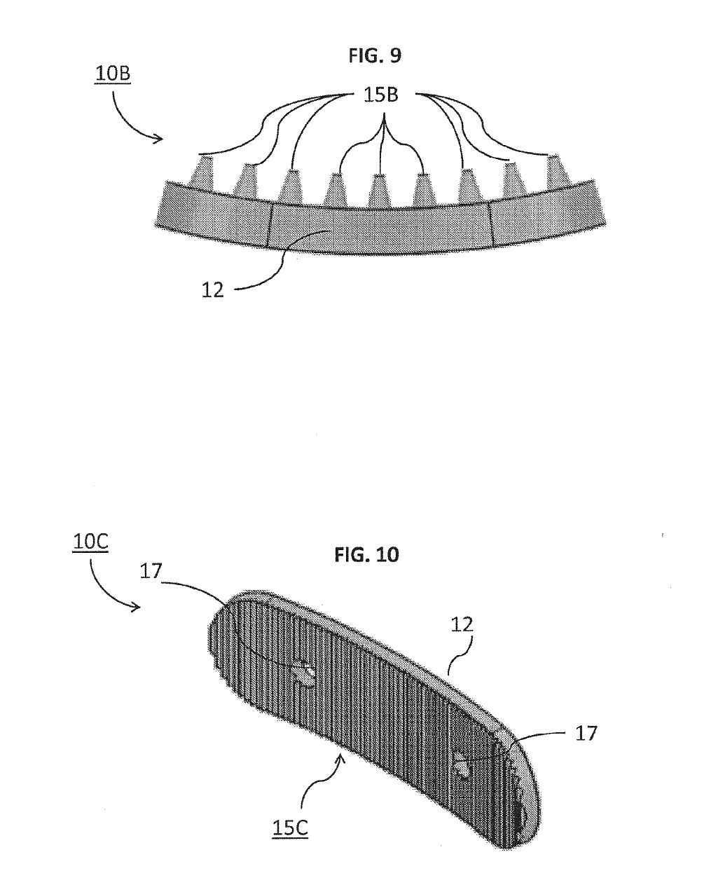

FIGS. 7-9 depict a fixation device 10B in accordance with one embodiment of the present disclosure, with modified fastening members 15B. Specifically, FIG. 7 depicts an upper perspective view of device 10B with similar eyelets 17 and base 12 as those shown for some other embodiments described herein, but instead of a pair of members, an array of members 15B are positioned on the lower surface of base 12. Members 15B may be in any form or shape operable to grip the tendon 20 and securely engage device 10B thereto. As can be seen, member 15B is relatively shorter in length than members 15/15A described above with reference to other embodiments, but device 10B is not limited. Member 15B may be longer as required and may be wider at its proximal 31B or distal 33B portion. While member 15B is depicted as being conical or tapered, device 10B is not limited and instead, member 15B may be an untapered, elongate member such as a pin with a sharpened point so that the array of members 15B may still securely engage with tendon 20 as needed. Finally, members 15B may be evenly spread across the lower surface of base 12 as depicted, may be selectively positioned in one or multiple regions (e.g. only be in the left-hand portion or be only in the left- and right-hand portion but not be positioned in the central portion).

In one implementation of various aspects of the present disclosure in accordance with some embodiments, after a suture anchor is inserted into the bone, a mattress type repair is performed where each end of the loaded suture is passed through tendon 20. The sutures are then passed through eyelets 17 of device 10B, and a knot may be tied over device 10B. Device 10B and its one or more members 15B may comprise a frictional cleated surface defined by one or more depicted friction inducing ridges or grooves which contacts tendon 20, wherein this surface may capture tendon 20 during the reattachment process. As can also be seen, device 10B may comprise a slight curvature to mimic the navicular bone, or other intended targets for tendon reattachment. In this respect, device 10 can more evenly distribute compression across its entire area to tendon 20.

While base 12 of device 10A is depicted being curved, device 10B is not limited and may be any shape or design as needed or required. Device 10B may therefore be substantially planar or may be flexible and capable of being both planar and curved so that device 10B is capable of conforming to the contours of associated tendon 20 during use.

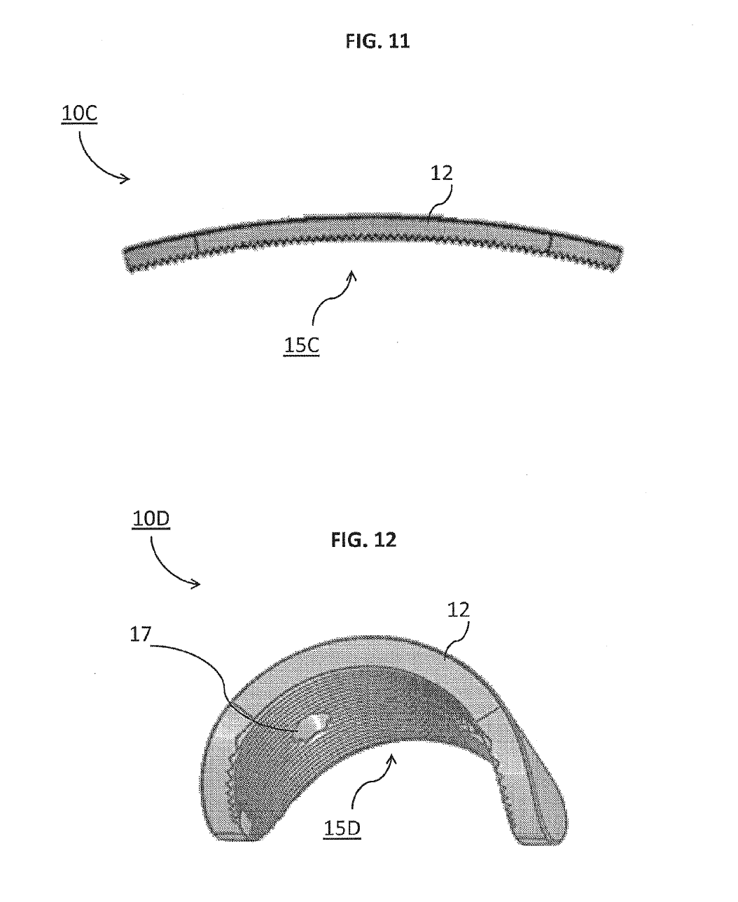

Turning to FIGS. 10-11, device 10C according to one embodiment of the present disclosure is shown. Specifically, FIG. 10 is a lower perspective view of device 10C clearly showing fastening surface 15C, whereas FIG. 11 is a side plan view of FIG. 10. Device 10C is similar to device 10B of FIGS. 7-9 but with different fastening member surfaces 15C. As can be seen, member 15C of device 10C may be constructed from a grip inducing pattern on the lower surface of base 12. In this respect, member 15C may also comprise a frictional cleated surface defined by the one or more depicted friction inducing ridges or grooves operable to contact tendon 20 during reattachment. In this respect, member 15C is capable of inducing more traction with tendon 20 as well as maximizing stability during reattachment. Specifically, member 15C is depicted with a plurality of ridges or grooves oriented in parallel with each other and orthogonal to the lateral side edges.

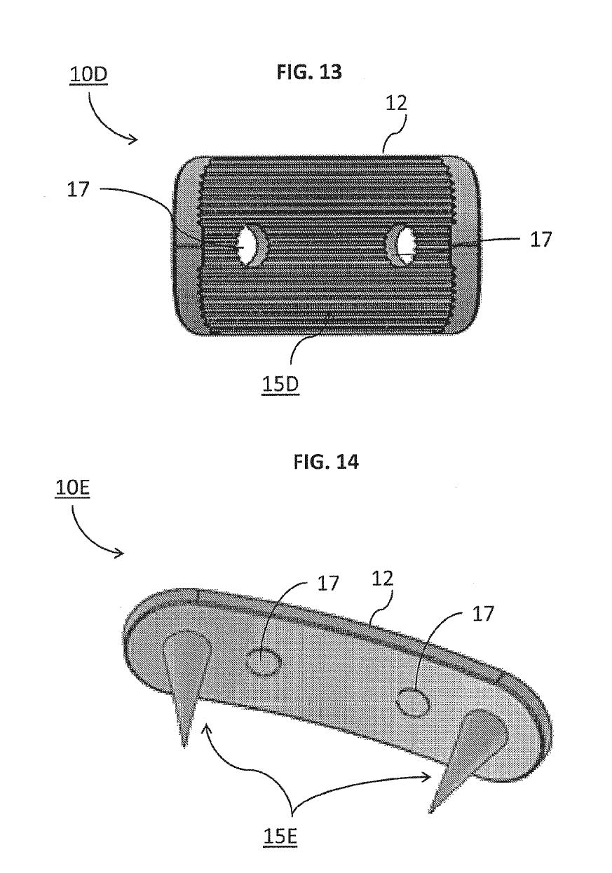

Any number of ridges or grooves may be provided as needed or required and each ridge or groove may have the same depth or may have differing depths. Further, each ridge or groove of member surface 15C may run diagonal or in parallel with respective lateral side edges of base 12 as seen in FIGS. 12-13, wherein member surface 15D runs parallel with lateral side edges of base 12. Optionally, the ridges or edges of member surface 15C may instead by a series concentric circles, ellipses, rectangles or any other shape instead of straight ridges as depicted. It should also be noted that the shape, width, curvature, and/or material can be altered to match specific tendon-bone regions as desired or needed. Any of the herein described fixation devices may have a low profile to avoid wound problems and/or skin irritation.

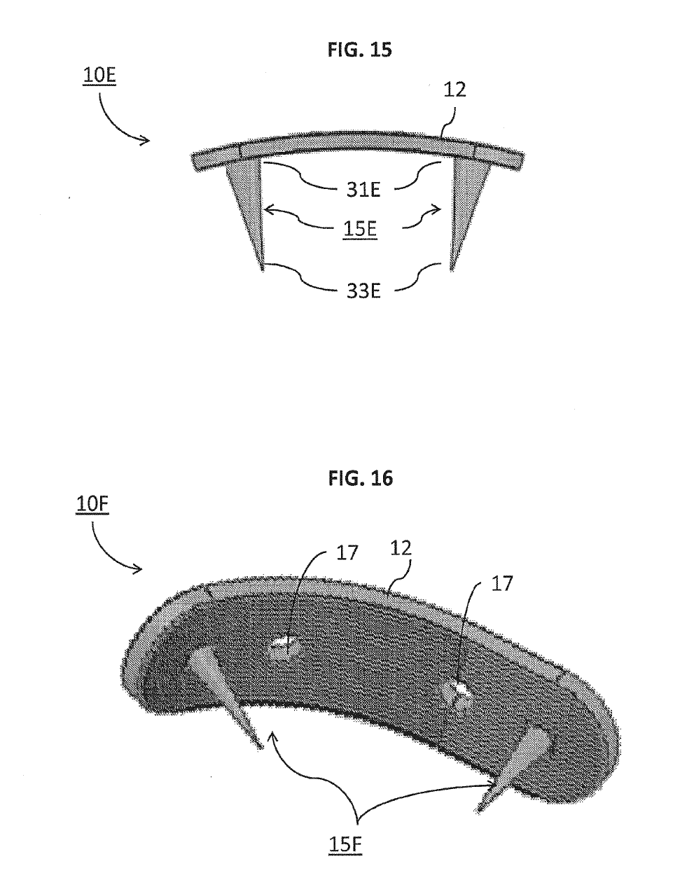

FIG. 14-15 depict a fixation device 10E according to one embodiment of the present disclosure, with some similarities to the device 10 shown in FIGS. 1-4. Device 10E and its constituent parts may be constructed from porous metal. Similar to previous embodiments, base 12 of device 10E may be curved as depicted or planar and be either rigid or flexible as need or required.

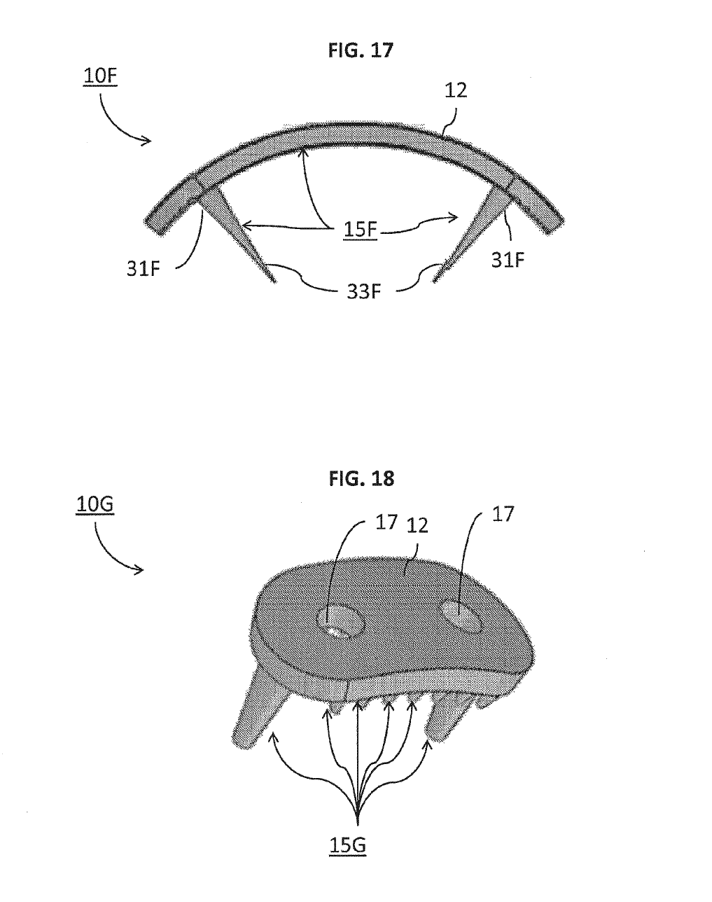

FIG. 16 depicts a lower perspective view of a fixation device 10F according to one embodiment of the present disclosure, with corresponding fastening members and fastening surface 15F disposed on underneath base 12. FIG. 17 depicts a side plan view of device 10F, wherein the curve, positioning, and orientation of members and surface 15F may be more clearly seen. In this embodiment, fastening members 15F may be a pair of members similar to those described in FIGS. 1-4 and fastening surface 15F may include one or more of the features previously described fastening surfaces including any number of ridges or grooves to facilitate engagement with tendon 20 during reattachment.

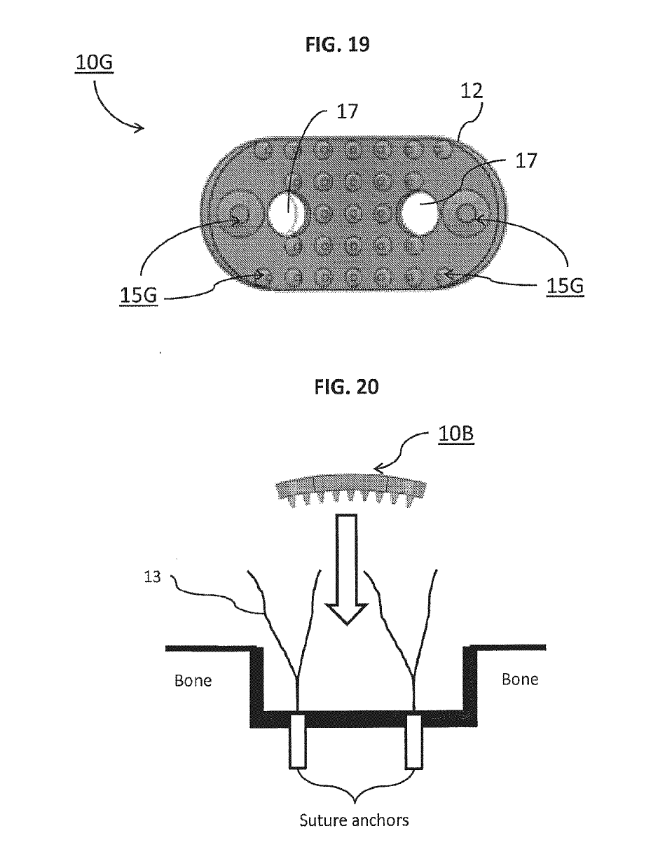

FIG. 18 depicts an upper perspective view of a fixation device 10G in according to one embodiment of the present disclosure, wherein device 10G comprises multiple different sized fastening members 15G. FIG. 19 depicts a bottom plan view of the device of FIG. 18 showing respective positions of the different sized fastening members 15G. More particularly, a pair of larger members 15G may be opposingly positioned on opposite halves of the lower surface of base 12. Disposed therebetween may be a plurality of relatively smaller members 15G. It should be noted that the diameters and/or lengths of each of the larger and smaller members 15G may differ as needed or required. Further, there may be only two different sized members 15G as depicted in FIG. 18 or there may be more than two different sized members. It should be noted that each of smaller members 15G may be distributed evenly between larger members 15G, or may selectively positioned in one or more regions or along a predetermined pattern (e.g. in rows, positioned in a series of diagonals, along the perimeter, etc.).

FIG. 20 depicts a schematic overview of a method of using a fixation device, in accordance with an embodiment of the present disclosure. The method is operable to be utilized with an implantation device 50 such as a drill or planer depending on the size of the herein described fixation devices that is implanted with tendon 20. For reference purposes, device 10B is depicted in FIG. 20 being used with the corresponding suture anchors, tendon 20, and bone region, but the method is not so limited and any of the herein described fixation devices may be replaced with device 10B. In this respect, to prepare a cancellous bed during the tendon and/or ligament repair of FIG. 20, a predetermined depth may be selected to correspond to the thickness of device 10B. The predetermined depth may depend, for example, on how much soft tissue coverage is possible. After one or a plurality of suture anchors are positioned at the predetermined depth of this prepared bed, the sutures may be passed through eyelets 17 of device 10B to be securely fastened thereto, for example, by being tied over the herein described fixation devices.

Eyelet 17 may be one, a pair, or more than two apertures or openings ranging in diameter sufficient to receive corresponding sutures. Eyelet 17 may have a diameter of 1 mm but the herein disclosed device 10 is not so limited and any sized opening of eyelet 17 may be provided sufficient to allow a suture to be passed therethrough. In this respect, an increase in surface area of compression may be exhibited versus the suture anchor as described more particularly below. Additionally, members 15 allow for penetration into tendon 20 as well as facilitate and/or promote bony ingrowth. Bone is also contemplated to be incorporated in the circumference of the described fixation devices thereby increasing the biologic healing and incorporation of the tendon.

As can be seen, devices 10 through 10G in accordance with various embodiments of the present disclosure may promote “footprint fixation” to restore the anatomic attachment of tendons or ligaments. As previously described, currently known techniques involve the use of bone tunnels or suture anchors which have known drawbacks. For example, among other problems, bone tunnels unnecessarily risk blowout, exhibit reduced or poor fixation with interference screws, loss of anatomic attachment. Similarly, suture anchors tend to suffer from inferior bony incorporation, questionable pull-out strength from the tendon, and experience problems with toggle.

The described fixation devices 10 through 10G in accordance with embodiments of the present disclosure, and their described uses, may also facilitate more robust bone to tendon and/or ligament healing of the FDL to the navicular in addition to nearly every subspecialty of orthopaedic surgery including, but not limited to, rotator cuff repair, tenodesis procedures (e.g. biceps), tendon transfers, Achilles and/or patellar tendon reattachment, ligament reattachment, and even as a reinforcement for ACL reconstruction. Further, the described fixation devices may permit tendon transfer even for obese patients requiring reconstruction. By achieving more stable fixation with the herein described fixation devices, earlier ranges of motion may be permitted, accelerating rehabilitation times and overall improved healing and function. Fixation devices according to some embodiments of the present disclosure may also allow patients to mobilize their repaired tendon earlier due to the increased strength of the interface. In this respect, herein described fixation devices in accordance with embodiments of the present disclosure may increase the contact area between the tendon and the bone thereby promoting healing with a decreased risk of re-injury.

Moreover, each of devices 10-10G may also comprise a coating with certain growth factors to facilitate healing. The coating may completely coat its respective fixation device, may partially coat its respective fixation device or may selectively coat said device in one or a plurality of predetermined treatment areas. Moreover, the coating may include a deposition or supply on device 10 or portions thereof with a medium that may comprise a pharmaceutically active substance, cells, fluids, biological fluids, drugs, gene therapy vectors, irrigation fluids, growth factors, nuclear medicine agents, antibiotics, anti-viral agents, contrast agents, chemotherapies, or other diagnostic or therapeutic agents. Each of the herein described fixation devices in accordance with embodiments of the present disclosure may also be operable to be visible on a medical imaging modality including but not limited to radiography, fluoroscopy, X-ray radiography, magnetic resonance imaging, medical ultrasonography or ultrasound, endoscopy, elastography, tactile imaging, thermography, tomography, echocardiography, medical photography and the like.

While members 15-15G may be spikes, ridges, barbs and/or cleats as depicted, it should be appreciated that other shapes and manners of fixation may be implemented so that the corresponding fixation device may be capable of exhibiting stability, friction interference, and osseous integration promotion. Members 15 through 15G may also include protrusions, tines, barbs, cones, studs, uprights, pedestals, pins, projections, fingers, or knurling.

Device 10 may be constructed from one material or may be constructed from a variety of materials including, but not limited to, one or more of the following: titanium, porous titanium, tantalum, biofoam®, polyether ether ketone, plastic, thermoplastic, polymer, metal, trabecular metal®, alloy, stainless steel, polylactic acid or polylactide, or silicon.

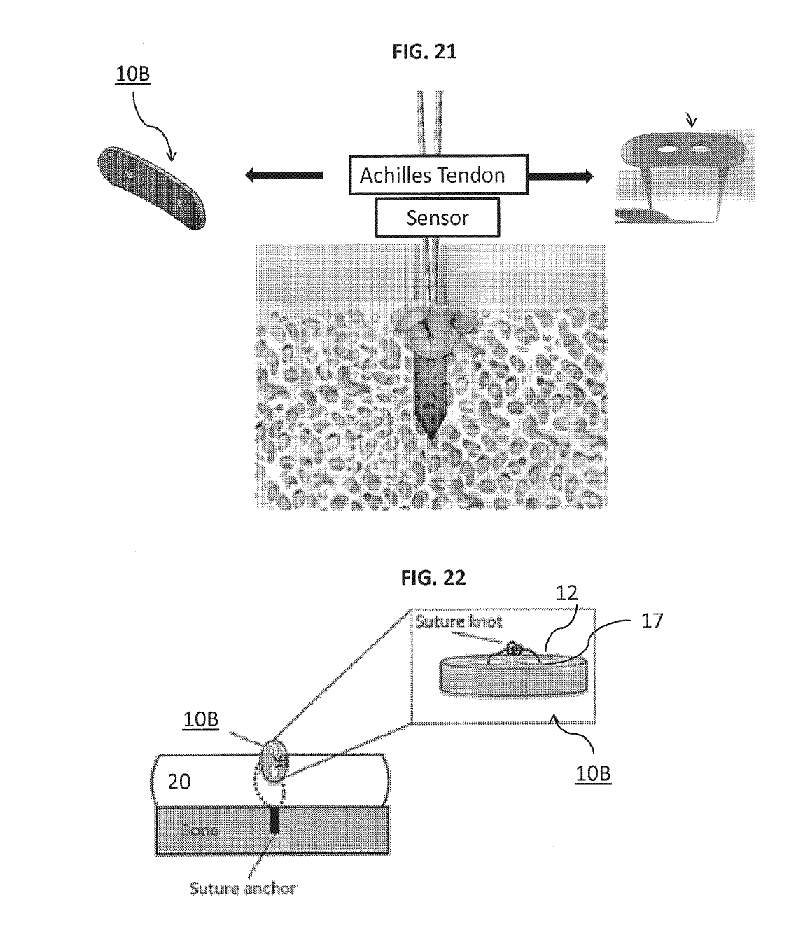

Various aspects of the present disclosure may be still more fully understood from the following description of some example implementations and corresponding results and the images of FIGS. 21-24B. Some experimental data are presented herein for purposes of illustration and should not be construed as limiting the scope of the disclosed technology in any way or excluding any alternative or additional embodiments.

A first example of implementing aspects of certain embodiments of the present disclosure and corresponding results will now be described with respect to FIG. 21, in which strength of compression delivered to human cadaver tendons and bone substitute was evaluated with respect to (i) a suture anchor alone; (ii) a suture anchor in combination with a suture fixation device similar to device 10B of FIGS. 10-11; and (iii) a suture anchor in combination with a porous metal implantation device similar to device 10 of FIGS. 1-4. Testing conditions on the cadaveric tendon included increased compressive forces and resistance to “cheese-cutter” effect using Instron® testing equipment (Instron Corp., Canton, Mass.) and Tekscan° sensors (Tekscan, Inc., Boston, Mass.), wherein loading was cyclical with 50% pull-out strength.

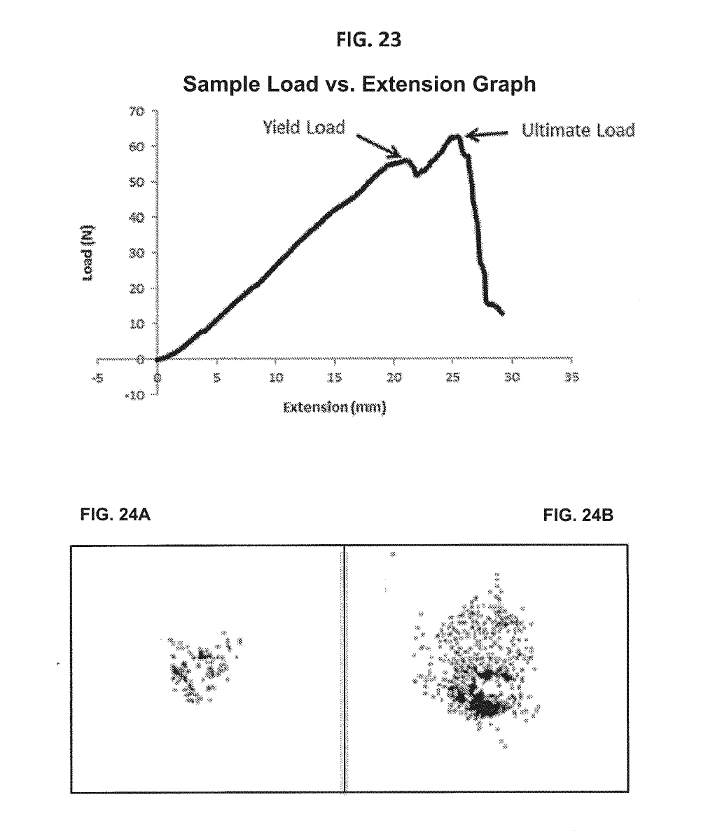

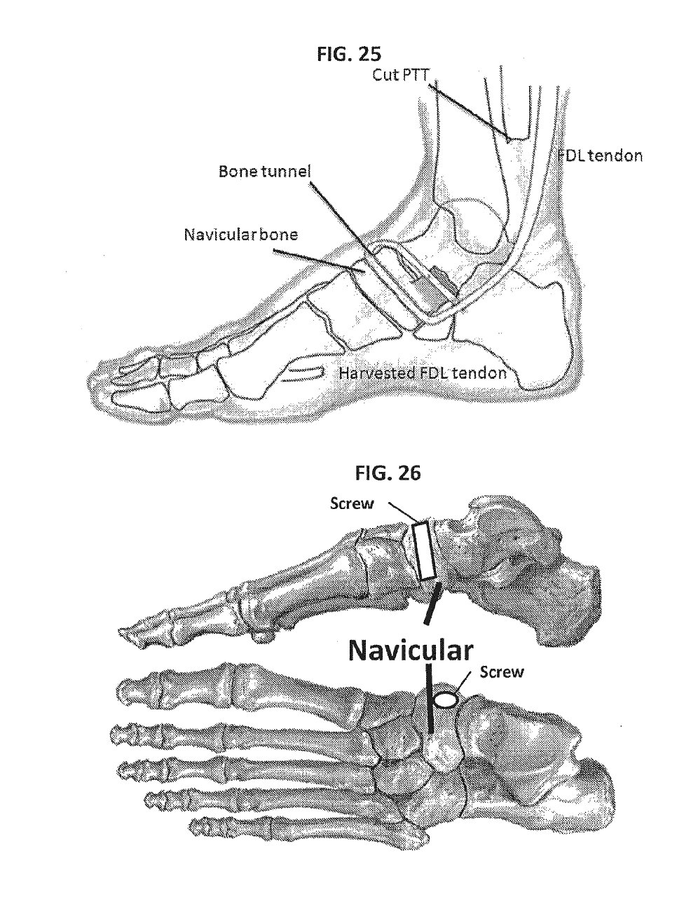

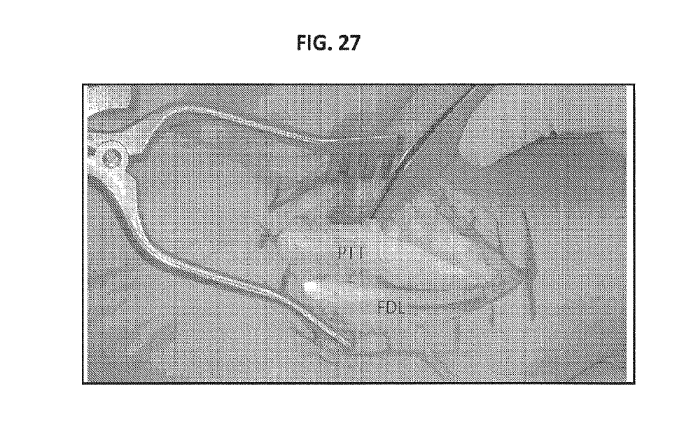

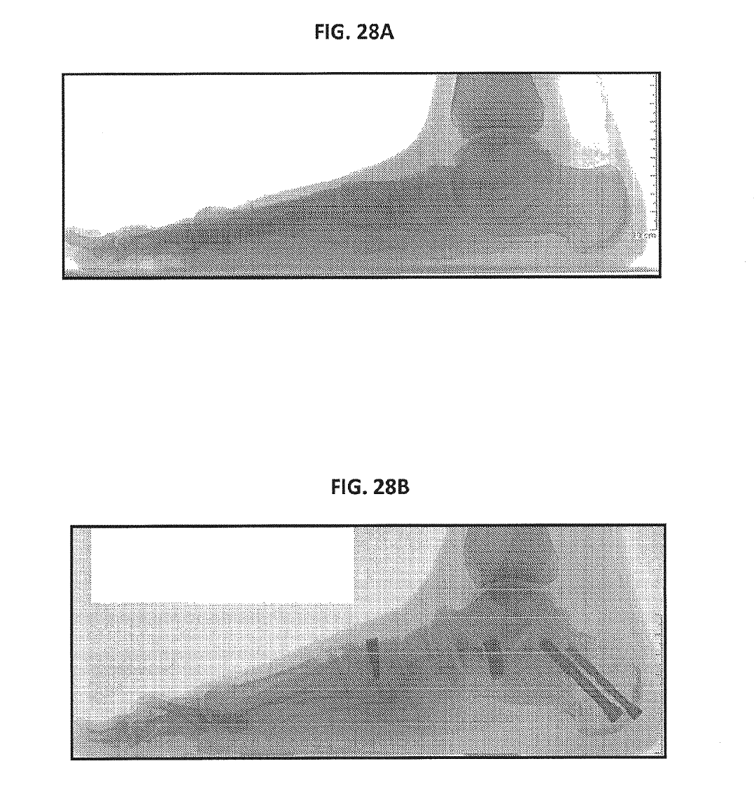

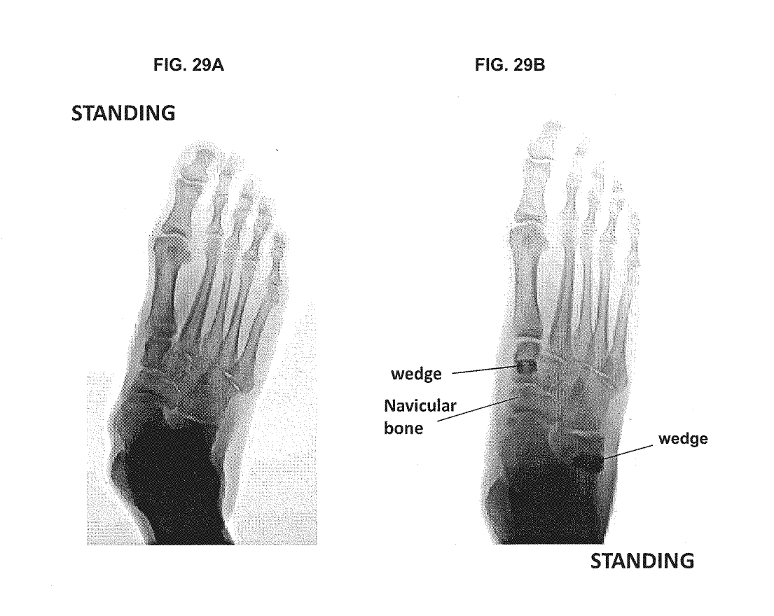

A second example of implementing aspects of some embodiments of the present disclosure and corresponding results will now be described. In this example implementation, twenty-four rabbit tendons were divided into control and device conditions based on the previously-described tendon reattachment method described with respect to FIGS. 10-11. Specifically, FIG. 22 depicts a schematic of the surgical setup used in this example, wherein tendon 20 was sutured to the bone block and the suture anchor was first inserted into the bone block, the sutures were passed through eyelets 17, and a surgical knot was finally tied on top of device 10B from FIGS. 10-11. Load and displacement data were acquired through failure tensile tests performed using an Instron® materials testing system (MTS Systems Corp, Eden Prairie, Minn.) in length-control mode with a speed of 0.5 mm/s. The yield load was defined as the maximum load value achieved before permanent damage to the tendon-bone block interface. The yield load was found by determining the best fit line of the linear portion of the graph and identifying any considerable deviations as depicted in FIG. 23, which graphically depicts load versus extension, illustrating the bimodal failure of the yield and ultimate loads. Ultimate tensile load was considered to be the peak force recorded. The specimens were secured onto the machine using Krackow type retention stitch methods. Similarly, FIGS. 24A and 24B depict binary images displaying the pressure area difference between the control (FIG. 24A) and device (FIG. 24B) conditions as recorded by the pressure indicating film. Pressure indicating film was used to capture the imprint of the tendon at its insertion site. It can be seen that the device condition (FIG. 24B) generated a larger contact area compared to the control condition (FIG. 24A).

Accordingly, the control condition was surgically repaired using a standard suture anchor, while the device condition combined the fixation device according to an embodiment of the present disclosure with the standard suture anchor. Specimens in each condition were subdivided and subjected to pressure film contact area testing, cyclic testing from 5 to 25 N for 50 cycles, and load-to-failure tensile testing. It was determined that contact area was significantly greater for the device condition (14.4±4.57 mm2) versus the control condition (3.77±2.24 mm2, P=0.002). The mean tendon lift off distance after cyclic loading was 0.19±0.09 mm in the device condition compared with 2.24±0.12 mm in the control condition (P<0.001). Load-to-failure tensile testing revealed a significantly greater yield load for the device condition (87.0±14.0 N) compared to the control condition (50.6±6.30 N, P=0.004). The use of the fixation device significantly increased the contact area between the tendon and the bone block substitute and allows for a better approximation of the anatomical footprint of the tendon thereby improving the engagement between the tendon and the bone and promote healing.

Therefore, this testing revealed that the use of a fixation devices as described above, in accordance with an embodiment of the present disclosure, significantly increased the contact area between tendon and bone, and the yield load. Additionally, the tendon lift off distance was significantly less following cyclic loading in the device condition compared to the control condition.

Turning to FIG. 30, an upper perspective view of fixation device 10 according to one embodiment of the present disclosure is depicted. In this embodiment, device 10 may be constructed from porous metal and be operable to securely engage with a tendon 20 (see e.g. FIG. 35 et seq.) through a friction and/or cleat engagement as between base portion 12 of device 10 and its pair of outwardly extending fastening members 15.

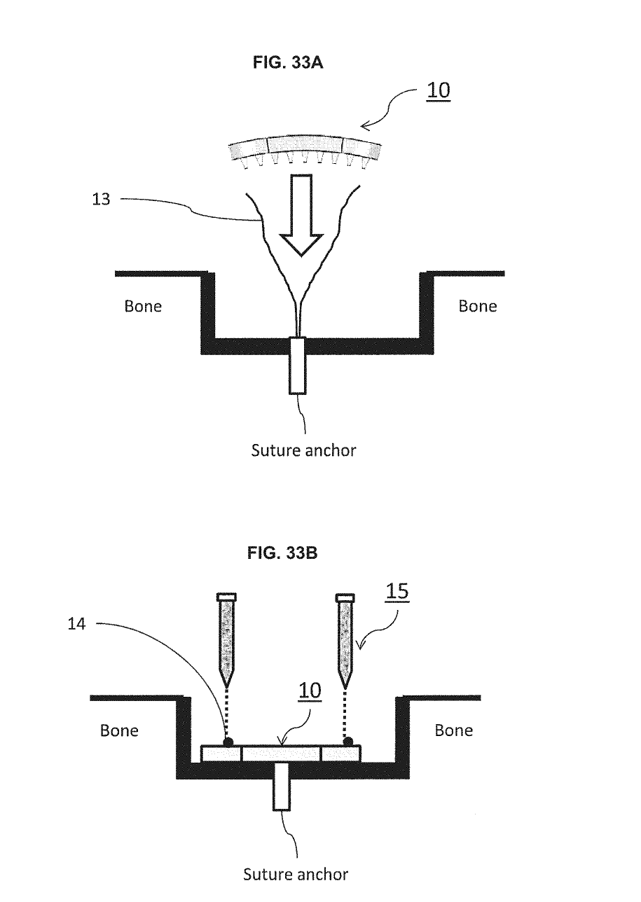

Each member 15 may be removably inserted through respective eyelets 17 formed on an upper surface of device 10. Each member 15 may therefore be elongate with an upper head 70 having a stop or washer surface operable to prevent member 15 from being inserted passed a predetermined depth with respect to base 12 and corresponding tendon 20 and/or bone region. A portion 73 of head 70 can be selectively removed for providing external access to one or more sutures 13 (see, e.g., FIGS. 33-35 et seq.) and/or corresponding suture knots. Portion 73 can be a cutout along the medial side of head 70 that allow room for suture 13 and member 15 to both fit through the eyelets 17. Head 70 may be removably or integrally formed with the remainder of member 15 as needed or required.

In practice, a repair may be performed on a tendon 20 using device 10 by securing one or more sutures 13 to eyelets 17. In certain embodiments (e.g. FIGS. 33A and 33B), suture knots 14 may also be formed before members 15 are assembled with device 10 to promote easy secure, precise and easy insertion. Sutures 13 may secure device 10 thereto by being tied or otherwise fastened with portion 70 of each member 15. Accordingly, after securing device 10 to tendon 20 with sutures 13, members 15 may be removably inserted through eyelets 17, perforating the subchondral bone thereby securing base 12 and sutures 13 to tendon 20. It is to be understood that each member 15 may be porous and may be used to perforate and capture the subchondral bone to promote osseous integration.

This novel engagement may be operable to stimulate bone ingrowth through and around tendon 20 while also promoting biologic healing for permanent tendon-bone fixation. Furthermore, device 10 is capable of reducing the “toggle” and rotation of device 10 once secured on top of tendon 20. As can be seen, device 10 can include multiple different sized or smaller fastening members 45 disposed on the tapered lower surface of base 12 adjacent and/or about fastening members 15. FIG. 31 depicts a bottom plan view of device 10 showing respective positions of the different sized fastening members 45 and 15. More particularly, a pair of larger members 15 may be opposingly and removably positioned on opposite halves of the lower surface of base 12. Disposed therebetween may be a plurality of relatively smaller members 45. It should be noted that the diameters and/or lengths of each of the larger and smaller members 15 and 45 may differ as needed or required. Further, there may be only two different sized members 15 as depicted in FIG. 30-31 or there may be more than two different sized members 15. It should be noted that each of smaller members 45 may be distributed evenly between larger members 15, or may selectively positioned in one or more regions or along a predetermined pattern (e.g. in rows, positioned in a series of diagonals, along the perimeter, etc.). Base 12 and members 45 may be formed as a one piece with removably insertable members 15 being relatively sharp to pierce the subchondral bone as well as pierce tendon 20 and/or go outside tendon 20.

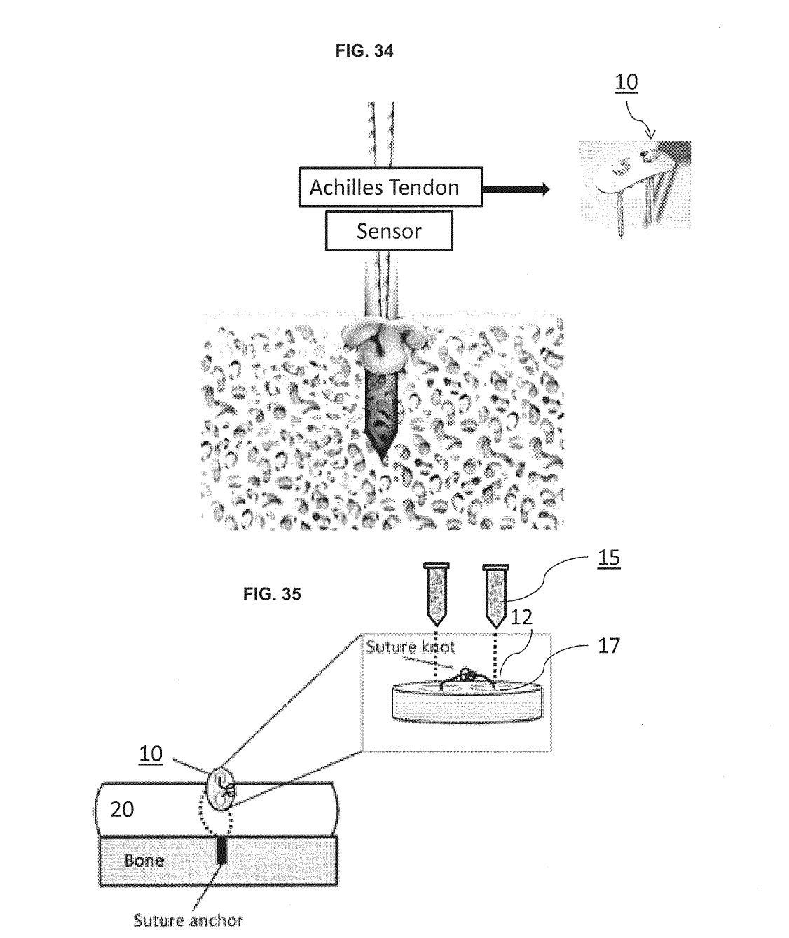

Device 10, base 12, members 15, eyelets 17, and/or members 45 (see e.g. FIG. 34 et seq.) may comprise any number of sizes, dimensions, contours, rigidity, shapes, flexibility and materials of any of the components or portions of components in the various embodiments of the device discussed throughout. Likewise each of the foregoing features of device 10 may be varied and utilized as desired or required including size, material properties, predetermined locations of device 10 and/or alignments of the various components of or with device 10 may vary as desired or required.

Each of the members 15 in this embodiment shown in FIG. 30-35 may commence with a proximal portion 31 and terminate at a distal pointed portion 33. It is understood that portion 31 may be wider than portion 33 so as to be able to secure device 10 to tendon 20 by, for example, a friction or cleat engagement by piercing the corresponding tendon 20 with each of members 15. Portion 33 may also include a tapered or pointed surface designed to perforate tissue and promote entry of member 15 through the corresponding bone region and tissue. Each member 15 may also include one or more multiple edges that are tapered entirely or partially between portions 31 to portion 33. One or more tapered edges of member 15 can be particularly advantageous as it can decrease irritation on the surgery site. Member 15 may also include a contoured surface that is relatively on or about portion 31 that decreases irritation with the overlying skin/soft tissues.

The external surface of member 15 may also include an external coating or texture to induce friction and secure fastening with corresponding bone region and tissue.

Base 12 may be flexible or resilient so that base 12 may be adjusted between one of a plurality of three dimensional arrangements or capable of conforming to the corresponding tendon 20 or ligament during implantation. Device 10 may also be relatively rigid and/or curved or contoured in a manner consistent with the corresponding tendon 20. Device 10 can also comprise a slight curvature to mimic the navicular bone, or other intended targets for tendon reattachment. In this respect, device 10 can more evenly distribute compression across its entire area to tendon 20. Members 15 as depicted may be outwardly extending spikes or barbs that are capable of piercing corresponding tissue and securely engaging device 10 in place during implantation. In some embodiments, members may be capable of perforating the bone to further stabilize device 10 during use. Preferably, device 10 may comprise porous metal or other materials to permit bony ingrowth.

Each member 15 of FIG. 30 may measure 5 mm in length and/or diameter but member 15 is not so limited and said dimensions of member 15 may be lesser or greater than 5 mm as needed or required by the desired application. Further, base portion 12 of device 10 may measure 8 mm in length and 3.5 mm in width though these dimensions may vary with either or both dimensions being relatively or substantially larger or smaller as needed or required. Shapes and sizes may depend on the intended bone targeted for tendon reattachment. Similarly, the number of eyelets can be increased for irregular or larger tendon footprints to increase fixation strength.

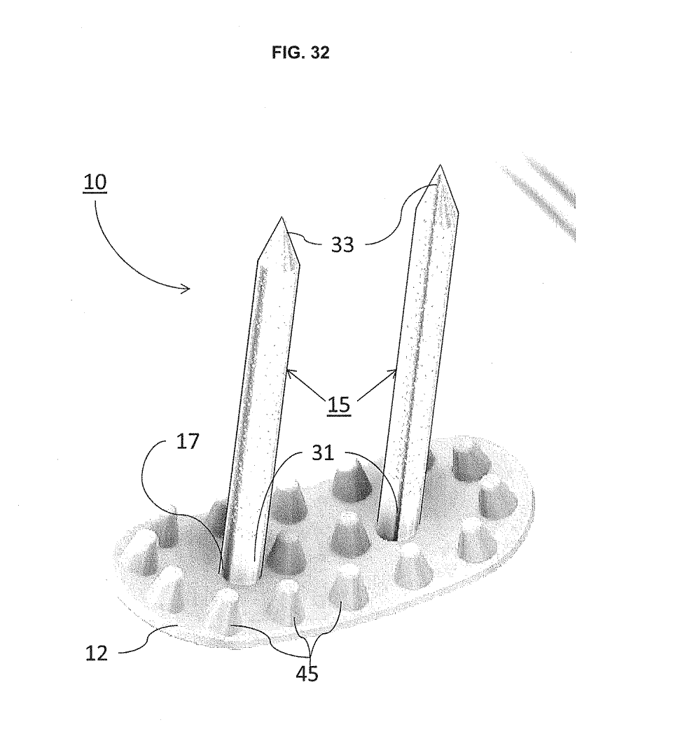

FIG. 31 depicts a lower plan view of the device 10 of FIG. 30 and FIG. 32 depicts a lower perspective view of device 10 of FIG. 30, wherein a pair of eyelets 17 are depicted on base 12 providing a portal or window between the upper and lower surfaces of base 12 to receive sutures 13 (see e.g. FIGS. 33-35 et seq.) or the like. While only two eyelets 17 are depicted in FIGS. 30-32, device 10 is not so limited and any number of eyelets 17 may be provided in any manner of shapes and sizes including circular, elliptical, rectangular or the like. Furthermore, spacing or positioning of eyelets 17 may be optimized to maximize compressive force delivered to tendon 20. Device 10 may accommodate multiple sutures 13 if allowed by the boney anatomy relative to tendon 20.

FIG. 31 depicts a bottom plan view of base 12 of device 10 with the illustrated positioning of eyelets 17 thereon. While FIG. 31 depicts only two members 15, device 10 is not limited and may include only one member 15 or any number of members 15 removably insertable through eyelets 17 and attachable with sutures 13 and/or knots 14 on base 12. Members 45 may also be positioned in one or more arrays or patterns on the lower surface 12 to for securely engage device 10 with corresponding tendon 20 prior to members 15 being removably secured thereon.

Members 45 may be in any form or shape operable to grip the tendon 20 and securely engage device 10 thereto. While members 45 are depicted as being conical or tapered, members 45 are not so limited and instead, each member 45 may be an untapered, elongate member such as a pin with a sharpened point so that the array of members 45 may still securely engage with tendon 20 as needed. Finally, members 45 may be evenly spread across the lower surface of base 12 as depicted, may be selectively positioned in one or multiple regions (e.g. only be in the left-hand portion or be only in the left- and right-hand portion but not be positioned in the central portion).

As can be seen with reference to FIGS. 33A-33B, device 10 when used with members 15 tendon 20 can produce greater and more secure distributed compressive force between tendon 20 and its insertion site thereby preventing the severing of tendon 20. Shape of base 12 of device 10 may range between circular, elliptical, rectangular, or any shape as desired or needed or some combination of shapes as is the case in exemplary base 12 of FIGS. 30-32.

FIG. 33A depicts one implementation of various aspects of the present disclosure in accordance with some embodiments, after a suture anchor is inserted into the bone. In FIG. 33A, a mattress type repair can be performed where each end of the loaded sutures 13 is passed through tendon 20. The sutures 13 are then passed through eyelets 17 of device 10, and a knot 14 may be tied over device 10. One or more members 15 may then be removably inserted through each eyelet 17 to secure device 10 to tendon 20 until head 70 of member 15 contacts the upper surface of device 10. When head 70 has contacted the upper surface of device 10, member 15 has obtained the precise depth of tendon 20 or its corresponding bone region. The external surface of members 15 and/or members 45 may comprise a frictional cleated surface defined by one or more depicted friction inducing ridges or grooves which contacts tendon 20, wherein this surface may capture tendon 20 during the reattachment process.

The lower surface of base 12 may be constructed from a grip inducing pattern. Base 12 may also comprise a frictional cleated surface defined by one or more depicted friction inducing ridges or grooves operable to contact tendon 20 during reattachment. In this respect, base 12 may be capable of inducing more traction with tendon 20 as well as maximizing stability during reattachment.

A plurality of ridges or grooves of base 12 may be oriented in parallel with each other and orthogonal to the lateral side edges. Any number of ridges or grooves may be provided as needed or required and each ridge or groove may have the same depth or may have differing depths. Further, each ridge or groove of base 12 may run diagonal or in parallel with respective lateral side edges of base 12. Grip inducing surfaces of base 12 may also include a series of concentric circles, ellipses, rectangles or any other shape. It should also be noted that the shape, width, curvature, and/or material can be altered to match specific tendon-bone regions as desired or needed. Any of the herein described embodiments of fixation devices in accordance with the present disclosure may have a low profile to avoid wound problems and/or skin irritation.

FIGS. 33A through 33B also depict a schematic overview of a method of using a fixation device 10, in accordance with an embodiment of the present disclosure. The method of FIGS. 33A through 33B is operable to be utilized with an implantation device such as a drill or planer depending on the size of the herein described fixation devices that is implanted with tendon 20. To prepare a cancellous bed during the tendon 20 and/or ligament repair of FIGS. 30-32, a predetermined depth may be selected to correspond to the thickness of device 10 and/or depth of member 15. The predetermined depth may depend, for example, on how much soft tissue coverage is possible. After one or a plurality of suture anchors are positioned at the prepared bed, the sutures 13 may be passed through eyelets 17 of device 10 to be securely fastened thereto, for example, by being tied over the herein described fixation devices. Members 15 may then be removably inserted through eyelets 17, fastened with sutures 13, and ultimately further secure device 10 to tendon 20. Sutures 13 may secure device 10 thereto by being tied into one or more knots 14 that can be attached to head 70 of member 15. It is to be understood that each member 15 may be porous and may be used to perforate and capture the subchondral bone to promote osseous integration.

Eyelet 17 may be one, a pair, or more than two apertures or openings ranging in diameter sufficient to receive corresponding sutures. Eyelet 17 may have a diameter of 1 mm but is not so limited and any sized opening of eyelet 17 may be provided sufficient to allow a suture to be passed therethrough. In this respect, an increase in surface area of compression may be exhibited versus the suture anchor as described more particularly below. Additionally, members 15 allow for penetration into tendon 20 as well as facilitate and/or promote bony ingrowth. Bone is also contemplated to be incorporated in the circumference of the described fixation devices thereby increasing the biologic healing and incorporation of the tendon.

As can be seen, devices 10 and/or members 15 disclosed herein in accordance with various embodiments of the present disclosure may promote “footprint fixation” to restore the anatomic attachment of tendons or ligaments. As previously described, currently known techniques involve the use of bone tunnels or suture anchors which have known drawbacks. For example, among other problems, bone tunnels unnecessarily risk blowout, exhibit reduced or poor fixation with interference screws, loss of anatomic attachment. Similarly, suture anchors tend to suffer from inferior bony incorporation, questionable pull-out strength from the tendon, and experience problems with toggle.

The described fixation devices 10 and/or members 15 in accordance with embodiments of the present disclosure, and their described uses, may also facilitate more robust bone to tendon and/or ligament healing of the FDL to the navicular in addition to nearly every subspecialty of orthopaedic surgery including, but not limited to, rotator cuff repair, tenodesis procedures (e.g. biceps), tendon transfers, Achilles and/or patellar tendon reattachment, ligament reattachment, and even as a reinforcement for ACL reconstruction. Further, the described fixation devices may permit tendon transfer even for obese patients requiring reconstruction. By achieving more stable fixation with the herein described fixation devices, earlier ranges of motion may be permitted, accelerating rehabilitation times and overall improved healing and function. Fixation devices according to some embodiments of the present disclosure may also allow patients to mobilize their repaired tendon earlier due to the increased strength of the interface. In this respect, herein described fixation devices in accordance with embodiments of the present disclosure may increase the contact area between the tendon and the bone thereby promoting healing with a decreased risk of re-injury.

Moreover, in some embodiments, device 10 and/or members 15 and 45 may also comprise a coating with certain growth factors to facilitate healing. The coating may completely coat its respective fixation device, may partially coat its respective fixation device or may selectively coat said device in one or a plurality of predetermined treatment areas. Moreover, the coating may include a deposition or supply on device 10 or portions thereof with a medium that may comprise a pharmaceutically active substance, cells, fluids, biological fluids, drugs, gene therapy vectors, irrigation fluids, growth factors, nuclear medicine agents, antibiotics, anti-viral agents, contrast agents, chemotherapies, or other diagnostic or therapeutic agents. Each of the herein described fixation devices in accordance with embodiments of the present disclosure may also be operable to be visible on a medical imaging modality including but not limited to radiography, fluoroscopy, X-ray radiography, magnetic resonance imaging, medical ultrasonography or ultrasound, endoscopy, elastography, tactile imaging, thermography, tomography, echocardiography, medical photography and the like.

While members 15 may be spikes, ridges, barbs and/or cleats as depicted, it should be appreciated that other shapes and manners of fixation may be implemented so that the corresponding fixation device may be capable of exhibiting stability, friction interference, and osseous integration promotion. Members 15 may also include protrusions, tines, barbs, cones, studs, uprights, pedestals, pins, projections, fingers, or knurling.

Device 10 may be constructed from one material or may be constructed from a variety of materials including, but not limited to, one or more of the following: titanium, porous titanium, tantalum, biofoam®, polyether ether ketone, plastic, thermoplastic, polymer, metal, trabecular metal®, alloy, stainless steel, polylactic acid or polylactide, or silicon.

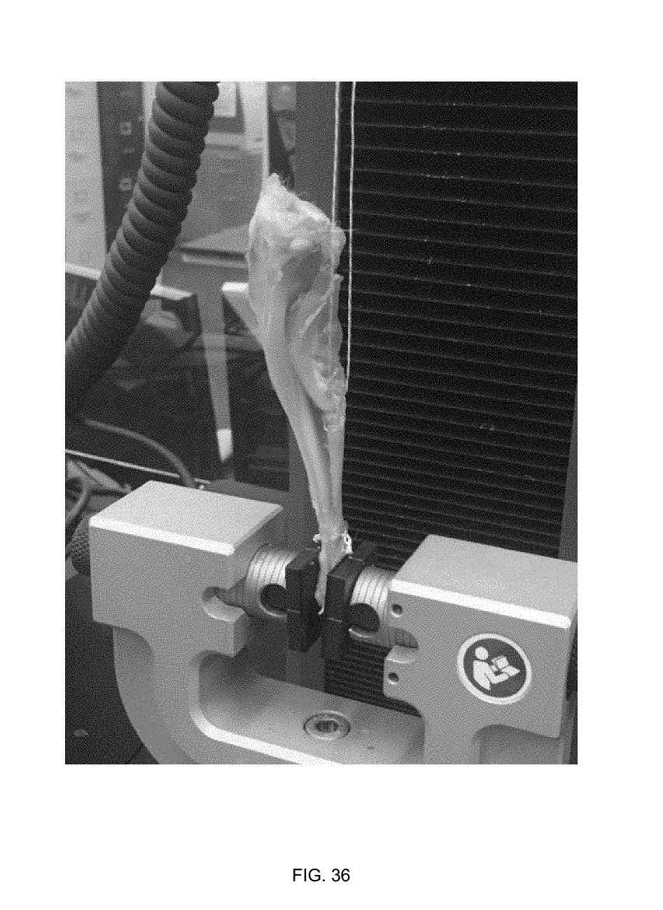

FIG. 36 is a photograph of an implanted device in accordance with the some embodiments of the present disclosure, involving a flexor digitorum longus transfer to the rabbit tibia.

### REFERENCES

Some references, including publications and non-patent literature references are cited in the disclosure provided herein and are listed individually below. The citation and/or discussion of such references is provided merely to clarify the description of the present disclosure and is not an admission that any such reference is “prior art” to any aspects of the present disclosure described herein. All references cited in this specification are incorporated herein by reference in their entireties and to the same extent as if each reference was individually incorporated by reference.

Burkhart et al., Biomechanical validation of load-sharing rip-stop fixation of tissue deficient rotator cuff tears. Am J Sports Med. 2014; 42(2):457-62

Barber et al., The relationship of suture anchor failure and bone density to proximal humerus location: a cadaveric study. Arthroscopy. 1997: 13(3);340-45

Yokoya et al., Tendon-bone insertion repair and regeneration using polyglycolic acid sheet in the rabbit rotator cuff injury model. Am J Sports Med. 2008:35(7):1298-1309

Kadonishi et al., Acceleration of tendon-bone healing in anterior cruciate ligament reconstruction using an enamel matrix derivative (EMD) in a rat model. J Bone Joint Surg Br. 2012;94(2):205-9

Moffat et al., Orthopedic interface tissue engineering for the biological fixation of soft tissue grafts. Clin Sports Med. 2009:28(1):157-76

### CONCLUSION

The specific configurations, choice of materials and the size and shape of various elements can be varied according to particular design specifications or constraints requiring a system or method constructed according to the principles of the disclosed technology. Such changes are intended to be embraced within the scope of the disclosed technology. The presently disclosed embodiments, therefore, are considered in all respects to be illustrative and not restrictive. The scope of the disclosure is indicated by the appended claims, rather than the foregoing description, and all changes that come within the meaning and range of equivalents thereof are intended to be embraced therein.

What is claimed is:

1. A tendon fixation method, comprising:
anchoring one or more suture anchor to a bone region;
securely engaging sutures to the one or more suture anchor;
fastening the sutures to a fixation device, the fixation device comprising a base with a plurality of openings to securely receive the sutures and one or more fastening members and/or fastening surfaces;
securing the fixation device to a tendon to thereby increase a resistance to rotation or axial motion; and
increasing contact area between the tendon and the bone region by distributing a compression force evenly from the base of the fixation device along a tendon region of the tendon.

2. The method of claim 1, wherein the suture anchors are anchored using an implantation device, the implantation device being a drill or a planer.

3. The method of claim 1, further comprising: promoting osseous integration of the tendon at an insertion site; and tying the sutures over the fixation device.

4. The method of claim 1, further comprising: preparing the bone region to a predetermined depth prior to anchoring the one or more suture anchor.

5. The method of claim 4, wherein the predetermined depth corresponds to a thickness of the fixation device.

6. The method of claim 1, wherein securing the fixation device to the tendon further comprises: stabilizing the fixation device by penetrating the tendon with the one or more fastening members and/or perforating the bone region with the one or more fastening members.

7. The method of claim 1, wherein the one or more fastening members are configured to be removably inserted into one or more of the plurality of openings in the base of the fixation device to a predetermined depth with respect to the bone region.

8. The method of claim 7, wherein each fastening member comprises an elongate member having distal and proximal portions, the distal portion comprising a piercing portion operable to pierce a tendon and the proximal portion comprising a washer surface wider than a remainder of the fastening member, the washer of the proximal portion operable to stop the fastening member once inserted to the predetermined depth.

9. The method of claim 1, wherein the fixation device is constructed from one or a combination of a porous metal, titanium, porous titanium, tantalum, biofoam, polyether ether ketone, plastic, thermoplastic, polymer, metal, trabecular metal, alloy, stainless steel, polylactic acid or polylactide, or silicon; or wherein the fixation device is bio-printed using biologically compatible materials including collagen or other substance allowing for the incorporation of the fixation device.

10. A tendon fixation device, comprising:
a base with a plurality of openings for detachably fastening with a plurality of sutures; and
a plurality of fastening members operable to securely engage with a tendon to increase a resistance of rotation or axial motion, wherein the base portion is capable of evenly distributing a compression force along a tendon region of the tendon.

11. The device of claim 10, wherein one or more of the fastening members extend outwardly from the base and are capable of perforating a bone region; or wherein one or more fastening members are configured to be removably inserted into one or more of the plurality of openings in the base of the fixation device and are capable of perforating a bone region.

12. The device of claim 10, wherein the base portion comprises a curvature.

13. The device of claim 12, wherein the curvature of the base portion is adjustable between a plurality of orientations ranging between substantially planar to substantially curved.

14. The device of claim 12, wherein the curvature corresponds to a contour of the bone region.

15. The device according to claim 10, wherein the fixation device is constructed from one or a combination of a porous metal, titanium, porous titanium, tantalum, biofoam, polyether ether ketone, plastic, thermoplastic, polymer, metal, trabecular metal, alloy, stainless steel, polylactic acid or polylactide, or silicon; or wherein the fixation device is bio-printed using biologically compatible materials including collagen or other substance allowing for the incorporation of the fixation device.

16. A fixation device, comprising:
a base with a plurality of openings for detachably fastening with a plurality of sutures; and
a fastening surface in communication with the base, the fastening surface being operable to induce traction with a tendon to increase a resistance of rotation or axial motion, wherein the base portion is capable of evenly distributing a compression along a tendon region of the tendon.

17. The device of claim 16, wherein the device is constructed from one or a combination of a porous metal, titanium, porous titanium, tantalum, biofoam, polyether ether ketone, plastic, thermoplastic, polymer, metal, trabecular metal, alloy, stainless steel, polylactic acid or polylactide, or silicon; or wherein the fixation device is bio-printed using biologically compatible materials including collagen or other substance allowing for the incorporation of the fixation device.

18. The device of claim 16, wherein the fastening surface comprises a frictional cleated surface defined by one or more friction inducing ridges or grooves operable to induce traction with the tendon.

19. The device of claim 16, wherein the fastening surface comprises ridges or grooves of differing depth.

20. The device of claim 16, wherein a curvature of the base portion is adjustable between a plurality of orientations ranging between substantially planar to substantially curved.

21. The device of claim 16, further comprising a plurality of outwardly extending fastening members, wherein the fastening members are capable of cleaving the tendon; wherein the fastening members extend outwardly from the base having a proximal portion adjacent the base and a distal portion that is capable of cleaving the tendon or wherein one or more fastening members are configured to be removably inserted into one or more of the plurality of openings in the base of the fixation device and are capable of cleaving the tendon and perforating the subchondral bone.

22. The device of claim 20, wherein at least two of the fastening members are capable of cleaving the tendon, perforating subchondral bone, and remaining fastening members are relatively smaller and capable of inducing traction with the tendon.
